(19) United States
(12) Patent Application Publication
(10) Pub. No.: US 2024/0304322 A1
(43) Pub. Date: Sep. 12, 2024

(54) PATIENT SUPPORT APPARATUS COMMUNICATION AND LOCATION SYSTEM

(71) Applicant: Stryker Corporation, Kalamazoo, MI (US)

(72) Inventors: Madhu Sandeep Thota, Portage, MI (US); Celso Henrique Farnese Pires Pereira, Portage, MI (US); Krishna Sandeep Bhimavarapu, Kalamazoo, MI (US); Madhu Thomas, London, Ontario (CA); Thomas Deeds, Seattle, WA (US); Kirby M. Neihouser, Portage, MI (US); Jerald A. Trepanier, Augusta, MI (US); Christopher P. Alvarez, Kalamazoo, MI (US)

(21) Appl. No.: 18/573,178

(22) PCT Filed: Sep. 15, 2022
(86) PCT No.: PCT/US2022/043587

§ 371 Date: Dec. 21, 2023

Related U.S. Application Data
(60) Provisional application No. 63/245,289, filed on Sep. 17, 2021.
(60) Provisional application No. 63/356,242, filed on Jun. 28, 2022.

(51) Int. Cl. G16H 40/67; A61G 7/05; H04W 12/06; H04W 12/63

(52) U.S. Cl. CPC G16H 40/67; H04W 12/06; H04W 12/63; A61G 7/05

(57) ABSTRACT

A patient support apparatus for supporting a patient communicates wirelessly with one or more devices and determines the relative position of the device(s) with respect to the patient support apparatus. A control system onboard the patient support apparatus receives identification data from the device(s) and uses the identification data to perform an authentication analysis of the device. Based on the authentication analysis, the control system determines an authorization level for the device and transmits different types of data to the device based on the authorization level. A plurality of locator nodes may be included on the patient support apparatus that are linked together by an embedded network. Synchronization messages are passed between the locator nodes over the embedded network to ensure the locator nodes have synchronized time. The authentication analysis and/or the use of encrypted communications may be based on the identity of the device and/or its location.

### BACKGROUND

The present disclosure relates to patient support apparatuses, such as beds, cots, stretchers, recliners, or the like. More specifically, the present disclosure relates to patient support apparatuses that are configured to communicate with one or more devices that are separate from, but within the vicinity of, the patient support apparatus.

### SUMMARY

According to the various aspects described herein, the present disclosure is directed to a patient support apparatus that includes a plurality of locator nodes for automatically determine the relative position of one or more devices that are positioned within the vicinity of the patient support apparatus. The plurality of locator nodes, in some embodiments, are time-synchronized with each other over an onboard embedded network. Communications between the patient support apparatus and the off-board device may be selectively encrypted depending upon an identification of the device and/or its location. Authentication of the device may also be dependent upon the identification and/or location of the device. In some aspects, the locator nodes communicate with device using ultra-wideband technology. Additional aspects of the present disclosure are described in more detail below.

According to one aspect of the present disclosure, a patient support apparatus is provided that includes a support surface, a plurality of locator nodes, an embedded network, and a control system. The support surface is adapted to support a patient thereon. The plurality of locator nodes each include a location transceiver and a location controller. The location transceiver is adapted to wirelessly communicate with a device positioned off-board the patient support apparatus and the location controller is adapted to determine a distance between the location transceiver and the device. The embedded network couples together the plurality of locator nodes. The control system is adapted to determine a location of the device relative to the patient support apparatus based on the distances determined by the location controllers, as well as to send time synchronization messages over the embedded network to the plurality of locator nodes. The time synchronization messages are adapted to allow each of the location controllers to determine a synchronized time measurement.

A patient support apparatus according to another aspect of the present disclosure includes a support surface, a plurality of locator nodes, a transceiver, and a control system. The support surface is adapted to support a patient thereon. The plurality of nodes each include a location transceiver and a location controller. The location transceivers are adapted to wirelessly communicate with a device positioned off-board the patient support apparatus. The location controllers are adapted to determine a distance between the location transceiver and the device. The transceiver is adapted to wirelessly communicate with the device. The control system is adapted to determine a location of the device relative to the patient support apparatus based on the distances determined by the location controllers. The control system is also adapted to determine if the location of the device meets a threshold condition, to receive identification data from the device, to perform an authentication analysis of the device if the threshold condition is met, and to not perform the authentication analysis of the device if the threshold condition is not met.

According to another aspect of the present disclosure, a patient support apparatus is provided that includes a support surface, a plurality of locator nodes, a transceiver, and a control system. The support surface is adapted to support a patient thereon. The locator nodes each include a location transceiver and a location controller. The location transceivers are adapted to wirelessly communicate with a device positioned off-board the patient support apparatus. The location controllers are adapted to determine a distance between the location transceiver and the device. The transceiver is adapted to wirelessly communicate with the device. The control system is adapted to determine a location of the device relative to the patient support apparatus based on the distances determined by the location controllers. The control system is further adapted to encrypt a first set of communications with the device after determining the location of the device, and to not encrypt a second set of communications with the device after determining the location of the device.

According to still other aspects of the present disclosure, the first set of communications includes communications between the location transceivers and the device that are used to determine the location of the device relative to the patient support apparatus, and the second set of communications includes communications between the transceiver and the device that are not used to determine the location of the device relative to the patient support apparatus.

According to some aspects of the present disclosure, the embedded network is a Controller Area Network (CAN).

In some aspects, the control system is adapted to determine a time difference of arrival of a signal transmitted from the device to each of the location transceivers.

The control system, in some aspects, is adapted to use the time difference of arrival to determine the location of the device relative to the patient support apparatus.

In some aspects, the patient support apparatus includes a motion control node adapted to control an actuator onboard the patient support apparatus. The motion control node is coupled to the embedded network and adapted to receive messages from the embedded network.

The patient support apparatus, in some aspects, further includes a control panel and a control panel node. The control panel includes a movement control adapted to be activated by a user and to control movement of a component of the patient support apparatus. The control panel node is in communication with the control panel and the embedded network, and it is adapted to transmit a motion control message to the motion control node in response to a user activating the movement control. The motion control message is sent with a lower priority than the time synchronization messages.

In some aspects, the location transceivers are ultra-wideband transceivers.

The device, in some aspects, is a wall unit affixed to a wall of a healthcare facility.

The patient support apparatus, in some embodiments, further includes a microphone adapted to convert voice signals from a patient onboard the patient support apparatus into audio signals. The control system is further adapted to transmit the audio signals to the wall unit. In some aspects, the control system is adapted to transmit the audio signals to the wall unit only after the control system has performed an authentication analysis and determined from the authentication analysis that the wall unit is an authentic device.

In some aspects, the patient support apparatus further includes a transceiver adapted to wirelessly communicate with the device and the control system is further adapted to determine if the location of the device meets a threshold condition, to receive identification data from the device, to perform an authentication analysis of the device if the threshold condition is met, and to not perform the authentication analysis of the device if the threshold condition is not met.

The transceiver, in some aspects, is a Bluetooth transceiver and each of the location transceivers are ultra-wideband transceivers.

In some aspects, the control system is further adapted to determine an authorization level for the device based on the authentication analysis.

In some aspects, the identification data includes a device certificate, and the control system is further adapted to transmit a patient support apparatus certificate to the device.

The device certificate, in some aspects, is received by the patient support apparatus in an encrypted form, and the control system is adapted to encrypt the patient support apparatus certificate before sending the patient support apparatus certificate to the device.

The control system, in some aspects, is adapted to share a random session key with the device if the control system authenticates the device certificate, and to use the random session key for subsequent communications with the device.

In some aspects, the control system uses the random session key for encoding communications with the device that are transmitted to the device via the transceiver. Alternatively, or additionally, the random session key may be used for encoding communications using the location transceivers.

In some aspects, the control system is adapted to not use the random session key for encoding communications with the device that are transmitted to the device via the location transceivers.

The patient support apparatus, in some aspects, further includes a network transceiver adapted to communicate with a remote server and the identification data includes a device certificate. The control system is further adapted to transmit the device certificate to the remote server as part of the authentication analysis.

The control system, in some aspects, is adapted to determine if the authorization level is a first level or a second level, and if the authorization level is the first level, the control system is adapted to accept data from the device but not transmit any sensor or patient information to the device. If the authorization level is the second level, the control system is adapted to both accept data from the device and to transmit sensor information, but not patient information, to the device.

The control system, in some aspects, is further adapted to determine if the authorization level is a third level, and if the authorization level is the third level, the control system is adapted to accept data from the device and to transmit patient information to the device.

The control system, in some aspects, is further adapted to use the identification data to determine the threshold condition.

In some aspects, the threshold condition is met if the device is positioned inside of a volume of space defined in a fixed relationship to the patient support apparatus, and the threshold condition is not met if the device is positioned outside of the volume of space.

In some aspects, the threshold condition is met if the patient support apparatus is positioned inside of a volume of space defined in a fixed relationship to the device, and the threshold condition is not met if the patient support apparatus is outside of the volume of space.

The control system may be further adapted to use the identification data to determine a size and/or shape of the volume of space.

In some aspects, the control system is adapted to encrypt communications between the device and the Bluetooth transceiver but not between the device and the location transceivers.

The control system, in some aspects, is adapted to receive the identification data from the device via at least one of the location transceivers.

The patient support apparatus, in some aspects, further includes a display, and the control system is adapted to display information on the display about the location of the device relative to the patient support apparatus.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of the patient support apparatus and a first type of wall unit that is used for automatically detecting the location of a patient support apparatus;

FIG. 5 is a block diagram of the patient support apparatus, wall unit, a plurality of tags, and computer network of FIG. 4;

FIG. 6 is a perspective view of the patient support apparatus and a second type of wall unit that is used for automatically detecting the location of the patient support apparatus;

FIG. 7 is a block diagram of the patient support apparatus, wall unit, and computer network of FIG. 6;

FIG. 8 is a block diagram of the patient support apparatus illustrating different thresholds used for determining the location of the patient support apparatus and one or more tagged devices;

FIG. 9 is a sequence diagram illustrating a server authentication algorithm that may be used by the patient support apparatuses, wall units, and/or tagged devices disclosed herein;

FIG. 10 is a sequence diagram illustrating an alternative authentication algorithm that may be used by the patient support apparatuses, wall units, and/or tagged devices disclosed herein;

FIG. 11 is a diagram illustrating different authorization levels that may be assigned to different devices; and

FIG. 12 is a diagram illustrating a pair of patient support apparatuses and various tags that may be automatically and/or manually associated with one of the two patient support apparatuses.

### DETAILED DESCRIPTION OF THE EMBODIMENTS

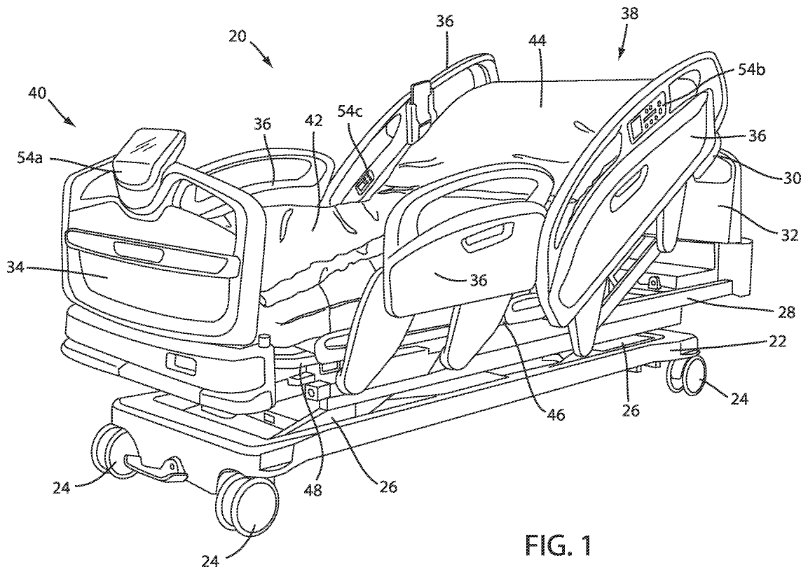

An illustrative patient support apparatus 20 according to an embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, an operating table, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,059 issued Apr. 6, 2010, to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes the functions and features discussed in greater detail below.

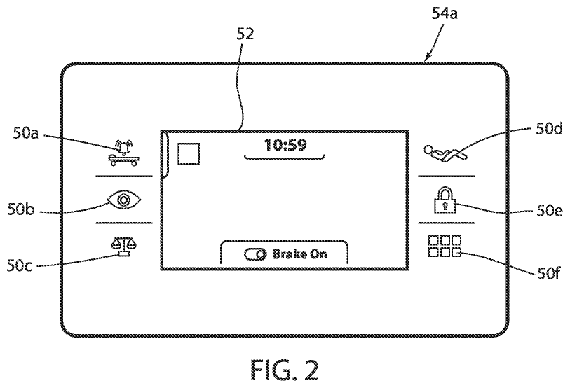

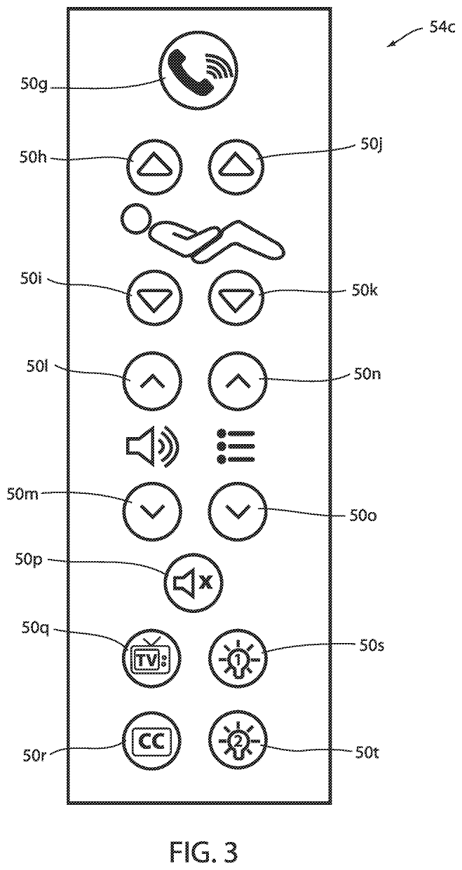

Patient support apparatus 20 further includes a plurality of control panels 54 that enable a user of patient support apparatus 20, such as a patient and/or an associated caregiver, to control one or more aspects of patient support apparatus 20. In the embodiment shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

Among other functions, controls 50 of control panel 54a allow a user to control one or more of the following: change a height of support deck 30, raise or lower head section 44, activate and deactivate a brake for wheels 24, arm and disarm one or more patient monitoring functions (discussed below), change various settings on patient support apparatus 20, view the current location of the patient support apparatus 20 as determined by the location detection system discussed herein, view what devices—if any—the patient support apparatus 20 has associated itself with, view the position of any tags that are positioned within the vicinity of the patient support apparatus 20 (as discussed in greater detail below), and perform other actions. One or both of the inner siderail control panels 54c also include at least one control that enables a patient to call a remotely located nurse (or other caregiver). In addition to the nurse call control, one or both of the inner siderail control panels 54c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state and/or the brightness level of these lights.

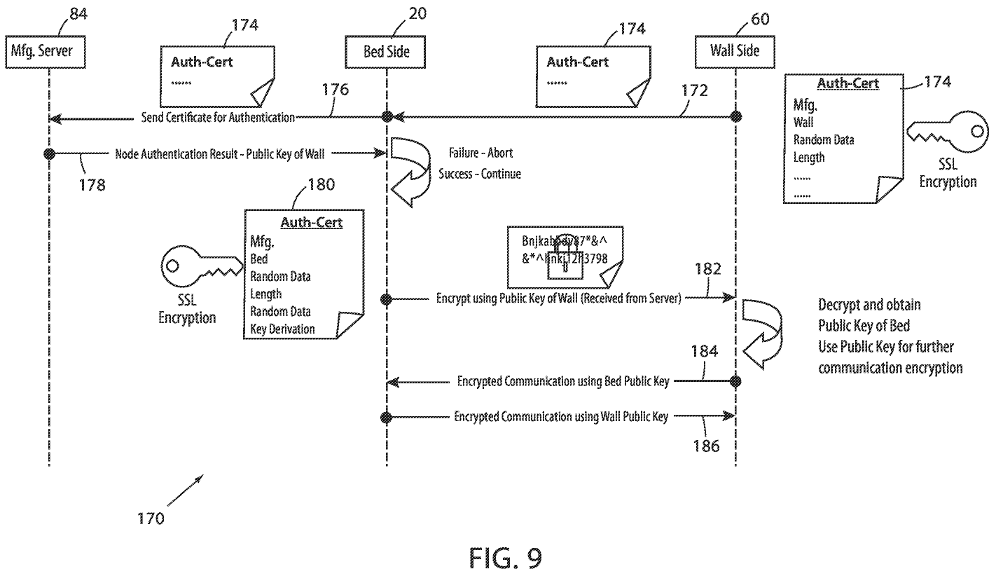

Control panel 54a includes a display 52 (FIG. 2) configured to display a plurality of different screens thereon. Surrounding display 52 is a plurality of navigation controls 50a-f that, when activated, cause the display 52 to display different screens on display 52. More specifically, when a user presses navigation control 50a, control panel 54a displays a patient monitoring screen on display 52 that includes one or more icons that, when touched, control one or more patient monitoring functions of patient support apparatus 20. In some embodiments, the patient monitoring screen presents the user with one or more controls for controlling an exit detection system. The exit detection system may include any of the functions, features, and/or components of exit detection system disclosed in commonly assigned U.S. patent application Ser. No. 16/917,004 filed Jun. 30, 2020, by inventors Sujay Sukumaran et al. and entitled PERSON SUPPORT APPARATUSES WITH ADJUSTABLE EXIT DETECTION ZONES, the complete disclosure of which is incorporated herein by reference. Other types of exit detection systems may, of course, be used. In other embodiments, the patient monitoring screen may present the user with additional, and/or alternative, patient monitoring options. One example of a patient monitoring screen that may be displayed on display 52 in response to a user pressing on control 50a is shown in FIG. 9 of commonly assigned U.S. patent application Ser. No. 63/245,279, filed Sep. 17, 2021, by inventors Jerry Trepanier et al. and entitled PATIENT SUPPORT APPARATUSES WITH PATIENT MONITORING, the complete disclosure of which is incorporated herein by reference.

When a user presses navigation control 50b (FIG. 2), control panel 54 displays a patient support apparatus monitoring control screen that includes a plurality of control icons that, when touched, control an onboard monitoring system that monitors one or more components, features, and/or other aspects of patient support apparatus 20. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20 for monitoring parameters of the patient support apparatus 20.

When a user presses navigation control 50c, control panel 54a displays a scale control screen that includes a plurality of control icons that, when touched, control the scale system of patient support apparatus 20. Such a scale system may include any of the same features and functions as, and/or may be constructed in any of the same manners as, the scale systems disclosed in commonly assigned U.S. patent application No. 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors that are utilized by an onboard exit detection system, in some embodiments. Other scale systems besides those mentioned above in the '254 and '954 applications may alternatively be included within patient support apparatus 20.

When a user presses navigation control 50d, control panel 54 displays a motion control screen that includes a plurality of control icons that, when touched, control the movement of various components of patient support apparatus 20, such as, but not limited to, the height of litter frame 28 and the pivoting of head section 44. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 54a displays a motion lock control screen that includes a plurality of control icons that, when touched, control one or more motion lockout functions of patient support apparatus 20. Such a motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

When a user presses on navigation control 50f, control panel 54a displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, location information, medical device association information, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 50f allows a user to navigate to a screen that enables a user to configure the communication settings between patient support apparatus 20 and one or more wall units (discussed more below). Examples of the type of communication settings that may be configured in this manner are disclosed in, and illustrated in FIGS. 9-15 of, commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION, the complete disclosure of which is incorporated herein by reference.

For all of the navigation controls 50a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f could alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls.

FIG. 3 illustrates one example of a patient control panel 54c that may be incorporated into patient support apparatus 20 and positioned at a location on patient support apparatus 20 that is convenient for a patient to access while supported on support deck 30, such as on an interior side of one of the siderails 36. Control panel 54c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise Fowler section 44 upwardly. A Fowler-down control 50i, when pressed by the patient, causes the motorized actuator to lower Fowler section 44 downwardly. A gatch-up control 50j, when pressed by the patient, causes another motorized actuator to raise a knee section of support deck 30, while a gatch-down control 50k causes the motorized actuator to lower the knee section of support deck 30.

A volume-up control 501, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 500, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20.

It will be understood that not only the number of controls 50 on control panel 54c, but also the functions of the controls 50 on control panel 54c, the layout of the controls 50 on control panel 54c, and/or other aspects of control panel 54c may be modified from what is shown in FIG. 3. In some embodiments, control panel 54c is implemented on a pendant controller that includes a cable that is plugged into a port on patient support apparatus 20. In other embodiments, one or more of the controls 50 of control panel 54c may be omitted, augmented, and/or split amongst other controls panels and/or locations. Still other manners of implementing control panel 54c are also possible.

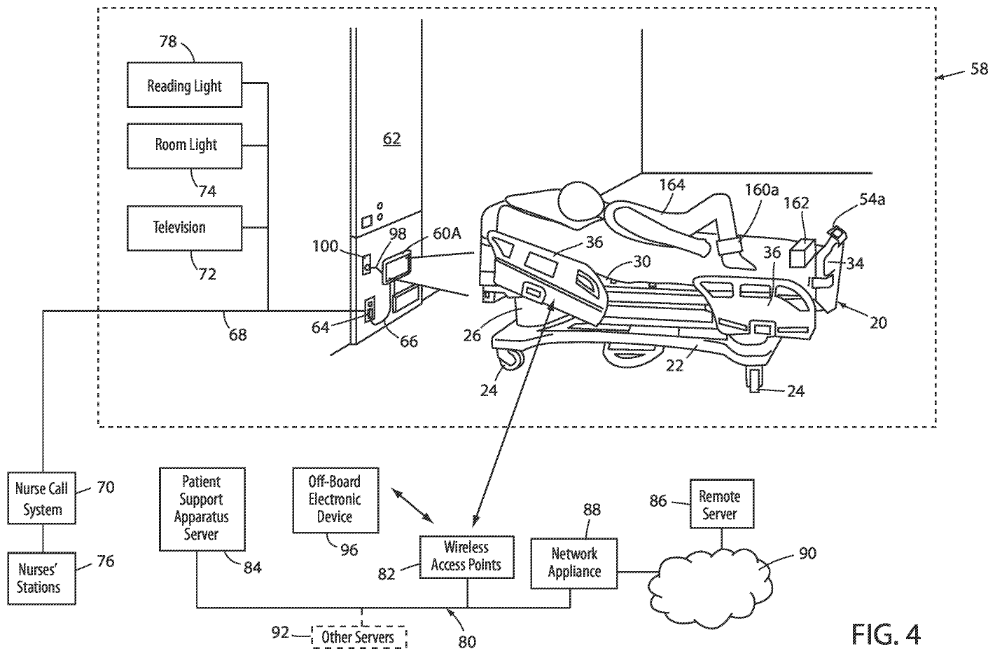

FIG. 4 illustrates a system for determining the location of patient support apparatus 20 when positioned within a room 58 of a conventional healthcare facility, such as, but not limited to, a hospital. This location detection system includes patient support apparatus 20, one or more wall units 60 (which may be of two types, 60a or 60b, as will be discussed in more detail below) and, in some aspects, a remote server, such as patient support apparatus server 84. Wall units 60 are positioned at known and fixed locations within the healthcare facility in which patient support apparatus 20 is positioned. As will be discussed in greater detail below, wall units 60 are adapted to determine how far away one or more patient support apparatuses 20 are from the wall unit 60 and/or they are adapted to allow patient support apparatuses 20 to determine how far away they are positioned from the wall unit 60.

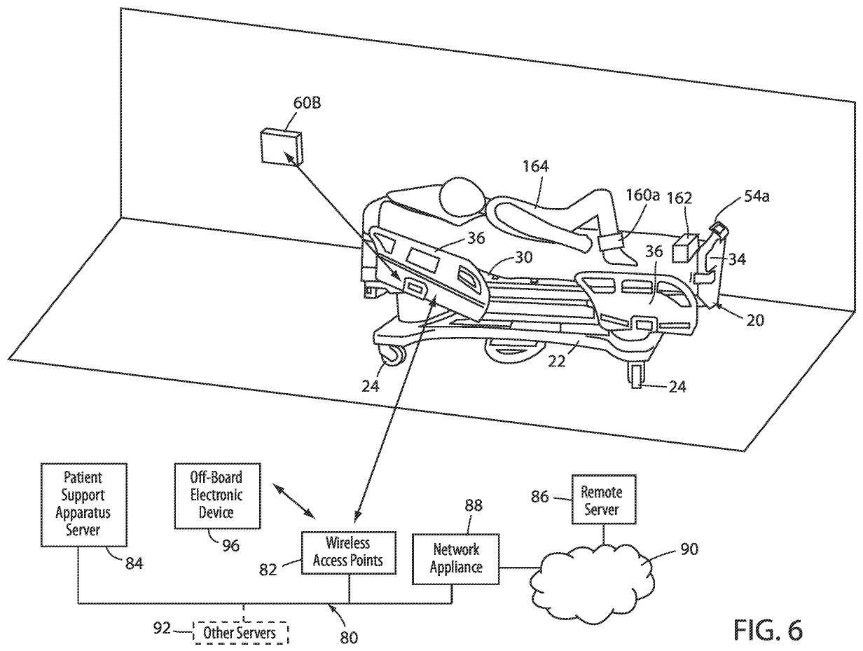

The location detection system described herein may utilize two different types of wall units 60: linked wall units 60a and unlinked wall units 60b. One example of a linked wall unit 60a is shown in FIG. 4. One example of an unlinked wall unit 60b is shown in FIG. 6. Wall units 60a and 60b differ from each other in that linked wall units 60a are adapted to communicate with a conventional communication outlet 64 that is typically built into one or more walls of a healthcare facility. That is, wall units 60a are communicatively linked to a conventional communication outlet 64. Unlinked wall units 60b are not adapted to communicate with such communication outlets 64, and are therefore not linked to a nearby communications outlet 64. Both wall units 60a and 60b are adapted to provide location information to a patient support apparatus. Linked wall units 60a, however, are also adapted to serve as a communication conduit for routing communications between patient support apparatus 20 and one or more devices and/or systems that are communicatively coupled to communication outlet 64 (e.g. room devices 72, 74, 78, and/or nurse call system 70). Unlinked wall units 60b, in contrast, are not necessarily adapted to serve as communication conduits between patient support apparatus 20 and any other electronic structures. In general, linked wall units 60a are typically positioned in patient rooms of the healthcare facility where one or more communication outlets 64 are typically present, while unlinked wall units 60b are typically positioned in locations outside of patient rooms, such as hallways, maintenance areas, and/or other areas. Unless explicitly stated otherwise, references to “wall units 60” made herein refer to both wall units 60a and 60b.

As shown in FIG. 4, linked wall units 60a are adapted to be mounted to a wall 62, such as a headwall of a patient room 58 within the healthcare facility. The headwall of a conventional healthcare facility room 58 typically includes a conventional communications outlet 64 physically integrated therein. Communications outlet 64 is adapted to receive a nurse call cable 66 that physically connects at its other end either to patient support apparatus 20 (not shown) or to wall unit 60a (shown in FIG. 4). In many healthcare facilities, communication outlet 64 includes a 37-pin connector, although other types of connectors are often found in certain healthcare facilities. As will be discussed in greater detail below, linked wall unit 60a and nurse call cable 66 allow patient support apparatus 20 to communicate with a nurse call system, and one or more room devices positioned within room 58.

Communication outlet 64 is electrically coupled to one or more cables, wires, or other conductors 68 that electrically couple the communication outlet 64 to a nurse call system 70 and one or more conventional room devices, such as a television 72, a room light 74, and/or a reading light 76. Conductors 68 are typically located behind wall 62 and not visible. In some healthcare facilities, conductors 68 may first couple to a room interface circuit board that includes one or more conductors 68 for electrically coupling the room interface circuit board to room device 72, 74, 78 and/or nurse call system 70. Still other communicative arrangements for coupling communication outlet 64 to nurse call system 70 and/or one or more room devices 72, 74, 78 are possible.

Nurse call cable 66 (FIG. 4) enables patient support apparatus 20 to communicate with nurse call system 70 and/or room devices 72, 74, 78. A patient supported on patient support apparatus 20 who activates a nurse call control (e.g. 50g; see FIG. 3) on patient support apparatus 20 causes a signal to be wirelessly sent from patient support apparatus 20 to linked wall unit 60a, which in turn conveys the signal via nurse call cable 66 to the nurse call system 70, which forwards the signal to one or more remotely located nurses (e.g. nurses at one or more nurse's stations 76). If the patient activates one or more room device controls (e.g. controls 501-t; see FIG. 3), one or more wireless signals are conveyed to linked wall unit 60a, which in turn sends appropriate signals via nurse call cable 66 to communication outlet 64 and the room device 72, 74, 78 that change one or more features of these devices (e.g. the volume, channel, on/off state, etc.).

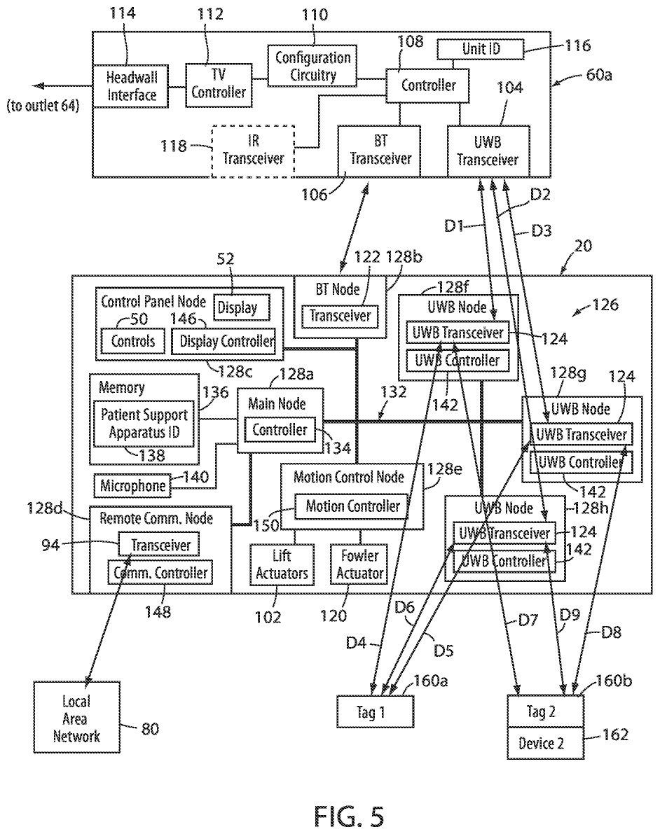

As is also shown in FIG. 4, patient support apparatus 20 is further configured to communicate with a local area network 80 of the healthcare facility. In the embodiment shown in FIG. 4, patient support apparatus 20 includes a wireless network transceiver 94 (FIG. 5) that communicates wirelessly with local area network 80. Network transceiver 94 is, in at least some embodiments, a WiFi transceiver (e.g. IEEE 802.11) that wirelessly communicates with one or more conventional wireless access points 82 of local area network 80. In other embodiments, network transceiver 94 may be a wireless transceiver that uses conventional 5G technology to communicate with network 80, one or more servers hosted thereon, and/or other devices. In some embodiments, network transceiver 94 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 94 is a wired transceiver that is adapted to allow patient support apparatus 20 to communicate with network 80 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 94 for communicating with network 80 via a wired connection and a wireless transceiver 94 for wirelessly communicating with network 80.

Patient support apparatus 20 is configured to communicate with one or more servers on local area network 80 of the healthcare facility. One such server is a patient support apparatus server 84. Patient support apparatus server 84 is adapted, in at least one embodiment, to receive status information from patient support apparatuses 20 positioned within the healthcare facility and distribute this status information to caregivers, other servers, and/or other software applications. As will be discussed in greater detail below, server 84 may also be configured to receive data from one or more tags that are positioned within one or more volumes of space defined around patient support apparatus 20. The tags may be attached to the patient and/or one or more devices. In some embodiments where data from devices is collected, the data from one or more of tags may be forwarded to one or more other servers 92 on network 80 (and/or one or more electronic devices 96), such as a caregiver assistance server and/or a caregiver assistance software application, as will also be discussed in greater detail below. Still further, in some embodiments, patient support apparatus 20 is utilized to determine the authenticity of one or more devices that are within communication range of patient support apparatus 20, as will be discussed in greater detail below.

In some embodiments, patient support apparatus server 84 is configured to communicate at least some of the patient support apparatus status data and/or the data from the tagged items that it receives from patient support apparatuses 20 to a remote server 86 that is positioned geographically remotely from the healthcare facility. Such communication may take place via a conventional network appliance 88, such as, but not limited to, a router and/or a gateway, that is coupled to the Internet 90. The remote server 86, in turn, is also coupled to the Internet 90, and patient support apparatus server 84 is provided with the URL and/or other information necessary to communicate with remote server 86 via the Internet connection between network 80 and server 86.

In some alternative embodiments, patient support apparatus 20 may be configured to communicate directly with one or more cloud-based servers, such as remote server 86, without utilizing patient support apparatus server 84. That is, in some embodiments, patient support apparatuses 20 may be configured to communicate directly with a remote server without relying upon any locally hosted servers (e.g. servers hosted on network 80). In one such embodiment, patient support apparatus 20 utilizes Microsoft's Azure could computing service to directly connect to one or more remote servers 86 without utilizing server 84. In some such embodiments, network appliance 88 may be a router configured to support such direct connections. Still other types of direct-to-cloud connections may be utilized with one or more of patient support apparatuses 20.

Patient support apparatus server 84 is also configured to determine the location of each patient support apparatus 20, or receive the location of each patient support apparatus 20 from the patient support apparatuses 20. In some embodiments, patient support apparatus server 84 determines the room number and/or bay area of each patient support apparatus 20 that is positioned within a room 58, as well as the location of patient support apparatuses 20 that are positioned outside of a room 58, such as, those that may be positioned in a hallway, a maintenance area, or some other area. In general, patient support apparatus server 84 may be configured to determine the position of any patient support apparatus 20 that is positioned within communication range of one or more wall units 60, as will be discussed in greater detail below.

It will be understood that the architecture and content of local area network 80 will vary from healthcare facility to healthcare facility, and that the example shown in FIG. 4 is merely one example of the type of network a healthcare facility may be employ. Typically, one or more additional servers 92 will be hosted on network 80 and one or more of them may be adapted to communicate with patient support apparatus server 84. For example, an electronic health record server will typically be present in any healthcare facility, and in some embodiments discussed herein, it will be in communication with patient support apparatus server 84 in order to receive patient data that is to be recorded in a patient's health record (e.g. weight readings taken from the scales built into patient support apparatuses 20; therapies provided to patients using a powered mattress 42 onboard patient support apparatuses 20, data from one or more tags and/or medical devices that are determined to be associated with the patient assigned to patient support apparatus 20, etc.). Local area network 80 will also typically allow one or more electronic devices 96 to access the local area network 80 via wireless access points 82. Such electronic devices 96 include, but are not limited to, smart phones, tablet computers, portable laptops, desktop computers, smart televisions, and other types of electronic devices that include a WiFi capability and that are provided with the proper credentials (e.g. SSID, password, etc.) to access network 80 (and, in at least some situations, patient support apparatus server 84).

Linked wall units 60a are adapted to wirelessly receive signals from patient support apparatus 20 and deliver the signals to communications outlet 64 in a manner that matches the way the signals would otherwise be delivered to communications outlet 64 if a conventional nurse call cable 66 were connected directly between patient support apparatus 20 and communications outlet 64. Linked wall units 60a are also adapted to transmit signals received from communications outlet 64 to patient support apparatus 20 via a Bluetooth transceiver 106 and/or a UWB transceiver 104 (FIG. 5). Thus, patient support apparatus 20 and linked wall unit 60a cooperate to send signals to, and receive signals from, communications outlet 64 in a manner that is transparent to communications outlet 64 such that outlet 64 cannot detect whether it is in communication with patient support apparatus 20 via a wired connection or it is in communication with patient support apparatus 20 via a wireless connection between patient support apparatus 20 and linked wall unit 60a (the latter of which is in wired communication with outlet 64). In this manner, a healthcare facility can utilize the wireless communication abilities of one or more patient support apparatuses 20 without having to make any changes to their existing communication outlets 64.

As noted, in addition to sending signals received from patient support apparatus 20 to communications outlet 64, linked wall units 60a are also adapted to forward signals received from communications outlet 64 to patient support apparatus 20. Linked wall units 60a are therefore adapted to provide bidirectional communication between patient support apparatus 20 and communications outlet 64. This bidirectional communication includes, but is not limited to, communicating command signals from any of controls 50 and/or from any of electronic devices 96 to corresponding room devices 72, 74, and/or 78 and communicating audio signals between a person supported on patient support apparatus 20 and a caregiver positioned remotely from patient support apparatus 20. The audio signals received by wall units 60 from a microphone on patient support apparatus 20 are forwarded to communications outlet 64 (for forwarding to nurse call system 70), and the audio signals of a remotely positioned nurse that are received at communications outlet 64 (from nurse call system 70) are forwarded to a speaker onboard patient support apparatus 20. Audio signals from a television 72 may also be communicated to the patient support apparatus via linked wall unit 60a.

Nurse call cable 66, in some embodiments, includes a conventional 37 pin connector on each end, one of which is adapted to be inserted into outlet 64 and the other one of which is adapted to be inserted into wall unit 60. Such 37 pin connections are one of the most common types of connectors found on existing walls of medical facilities for making connections to the nurse call system 70 and room devices 72, 74, and 78. Linked wall unit 60a and nurse call cable 66 are therefore configured to mate with one of the most common type of communication outlets 64 used in medical facilities. Such 37 pin connectors, however, are not the only type of connectors, and it will be understood that linked wall units 60a can utilize different types of connectors that are adapted to electrically couple to different types of nurse call cables 66 and/or different types of communication outlets 64. One example of such an alternative communications outlet 64 and cable 66 is disclosed in commonly assigned U.S. patent application Ser. No. 14/819,844 filed Aug. 6, 2015 by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of communication outlets 64 and corresponding connectors may be utilized.

Linked wall unit 60a (FIG. 4) also includes an electrical cord 98 having a plug positioned at a far end that is adapted to be inserted into a conventional electrical outlet 100. Electrical cord 98 enables linked wall unit 60a to receive power from the mains electrical supply via outlet 100. It will be appreciated that, in some embodiments, linked wall unit 60a is battery operated and cord 98 may be omitted. In still other embodiments, linked wall unit 60a may be both battery operated and include cord 98 so that in the event of a power failure, battery power supplies power to linked wall unit 60a, and/or in the event of a battery failure, electrical power is received through outlet 100. Unlinked wall units 60b may also include a battery, electrical cord, or both.

In addition to any of the structures and functions described herein, wall units 60a (and 60b) are configured to communicate location data to patient support apparatus 20 that enables patient support apparatus 20 and/or patient support apparatus server 84 to determine the location of patient support apparatus 20 within the healthcare facility. In general, such location determination is carried out by wall units 60 sending a unique wall identifier (ID) to one or more patient support apparatuses 20 that are positioned in close proximity thereto. Patient support apparatus 20 and/or wall unit 60 are further adapted to determine their position relative to the other. The combination of the patient support apparatus's relative position and the ID of the wall unit 60 is used either locally by patient support apparatus 20 to determine its position within the healthcare facility, or used remotely by server 84 to determine the position of the patient support apparatus 20 within the healthcare facility.

If determined remotely, patient support apparatus 20 may send its relative position information and/or the ID of the wall unit 60 (and its own unique patient support apparatus ID 130 (FIGS. 5 & 7) to server 84. Server 84 includes a table of all of the locations of the wall units 60 (which may be generated via a surveying operation during the installation of wall units 60), and it uses that table to correlate the patient support apparatus IDs 130 and the wall unit IDs it receives to specific locations within the healthcare facility. Thus, if a particular patient support apparatus 20 (with a particular ID 130) sends a wall unit ID that corresponds to room 430, server 84 determines that that particular patient support apparatus 20 is currently located in room 430 (and/or in a specific position relative to that wall unit 60). Server 84 determines that the particular patient support apparatus 20 is in the same room (room 430 in this example) as the wall unit 60 because each patient support apparatus 20 is configured to associate itself with a particular wall unit 60 when it is positioned within relatively close proximity thereto. Further details of this location determination process are described below, as well as in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

Wall units 60 may also perform additional functions. In some embodiments, wall units 60 (and/or patient support apparatuses 20) may perform any of the functions performed by the headwall units 76 (and/or patient support apparatuses) disclosed in commonly assigned U.S. patent application Ser. No. 16/215,911 filed Dec. 11, 2018, by inventors Alexander Bodurka et al. and entitled HOSPITAL HEADWALL COMMUNICATION SYSTEM, the complete disclosure of which is incorporated herein by reference. In some embodiments, wall units 60 and/or patient support apparatuses 20 may also, or alternatively, perform any of the same functions performed by the headwall interfaces 72 and/or patient support apparatuses disclosed in commonly assigned U.S. patent application Ser. No. 16/193,150 filed Nov. 16, 2018, by inventors Alexander Bodurka et al. and entitled PATIENT SUPPORT APPARATUSES WITH LOCATION/MOVEMENT DETECTION, the complete disclosure of which is also incorporated herein by reference. In still other embodiments, wall units 60 and/or patient support apparatuses 20 may also, or alternatively, perform any of the same functions performed by the headwall units 66 and patient support apparatuses disclosed in commonly assigned U.S. patent application Ser. No. 16/217,203 filed Dec. 12, 2018, by inventor Alexander Bodurka et al. and entitled SMART HOSPITAL HEADWALL SYSTEM, the complete disclosure of which is incorporated herein by reference.

In some embodiments, wall units 60 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION, the complete disclosure of which is incorporated herein by reference.

In some embodiments, wall units 60 and/or patient support apparatuses 20 may also be constructed to include any or all of the functionality of the headwall units and/or patient support apparatuses disclosed in commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION, the complete disclosure of which is also incorporated herein by reference.

Still further, in some embodiments, wall units 60 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference.

In some embodiments, patient support apparatus 20 and/or patient support apparatus server 84 may include any or all of the functionality of the patient support apparatuses and/or patient support apparatus servers described in any of the aforementioned commonly assigned U.S. patents and/or patent applications.

FIG. 5 depicts a block diagram of patient support apparatus 20 and linked wall unit 60a. Linked wall unit 60a includes an ultra-wideband transceiver 104, a Bluetooth transceiver 106, a wall unit controller 108, configuration circuitry 110, a television controller 112, a headwall interface 114, a unit ID 116, and, in some embodiments, an infrared transceiver 118. Bluetooth transceiver 106 is adapted to communicate with a Bluetooth transceiver 122 onboard patient support apparatus 20 using RF waves in accordance with the conventional Bluetooth standard (e.g. IEEE 802.14.1 and/or the standard maintained by the Bluetooth Special Interest Group (SIG) of Kirkland, Washington, USA). In some embodiments, transceivers 106 and 122 utilize Bluetooth Low Energy communications.

Ultra-wideband transceiver 104 is adapted to communicate with one or more ultra-wideband transceivers 124 positioned onboard patient support apparatus 20. Transceiver 104 is adapted to determine distances D1-D3 between itself and transceivers 124 of patient support apparatus 20. Alternatively, or additionally, transceiver 104 may be adapted to allow transceivers 124 onboard patient support apparatus 20 to determine distances D1-D3. In some embodiments, transceivers 104 and 124 use time of flight (TOF) computations to determine distance D1. In other embodiments, transceivers 104 and 124 may utilize other techniques for determining distances D1-D3, either in addition to, or in lieu of, TOF computations. In some embodiments, transceivers 104, 124 may also determine an angle between patient support apparatus 20 and wall unit 60 using angular information derived from antenna arrays positions onboard transceivers 104, 124, or by using other techniques.

In some embodiments, transceivers 104, 124 are implemented as any of the Trimension™ ultra-wideband modules available from NXP Semiconductors of Austin, Texas. These modules include, but are not limited to, the Trimension™ UWB modules ASMOP1BO0N1, ASMOP1CO0R1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement transceivers 104 and 124.

Wall unit controller 108 is adapted to control the operation of transceivers 104, 106, configuration circuitry 110, TV controller 112, headwall interface 114, and, if included, IR transceiver 118 (FIG. 5). When infrared transceiver 118 is included, it may be included to provide backwards compatibility with patient support apparatuses 20 that are not equipped with a UWB transceiver 124. That is, some healthcare facilities may include one or more patient support apparatuses that are not equipped with any UWB transceivers 124, but that do include an IR transceiver that is adapted to communicate with IR transceiver 118. When linked wall unit 60a includes IR transceiver 118, it is able to communicate its unit ID 116 to such patient support apparatuses via IR transceiver 118, which is a short range transceiver that is configured to only communicate with an adjacent patient support apparatus when the patient support apparatus is nearby (e.g. without about five feet or so). Such an adjacent patient support apparatus 20 then communicates the received wall unit ID 116 along with its own unique ID 130 (FIGS. 5 & 7) to server 84 which, as noted previously, is able to correlate the wall unit ID 116 to a particular location with the healthcare facility. In this manner, server 84 is able to use linked wall units 60a determine the location of versions of patient support apparatuses 20 that don't have a UWB transceiver 104, but that do have an IR transceiver.

Headwall interface 114 is adapted to change the electrical state of one or more pins that are in electrical communication with communication outlet 64 (via cable 66). Headwall interface 114 changes these electrical states in response to instructions from controller 108. For example, if an exit detection system onboard patient support apparatus 20 detects a patient exit, control system 126 sends an exit alert signal to linked wall unit 60a and controller 108 responds by instructing headwall interface 114 to change the electrical state of at least one pin that is used to signal an exit alert (or a generic priority alert) to the nurse call system 70 via communications outlet 64. In some embodiments, headwall interface 114 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCIING, the complete disclosure of which is incorporated herein by reference. Alternatively, or additionally, headwall interface 114 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Linked wall unit 60a may also be configured to perform any of the functions of the headwall units 94 disclosed in the above-mentioned '778 patent application.

Configuration circuitry 110 and TV controller 112 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked wall unit 60a may be configured to perform any of the functions of the headwall units 144 disclosed in the aforementioned '508 patent application.

Patient support apparatus 20 includes a control system 126 comprising a plurality of nodes 128a-h coupled together by an embedded network 132. Nodes 128 include a main control node 128a, a Bluetooth node 128b, a control panel node 128c, a remote communications node 128d, a motion control node 128e, and three ultra-wideband (UWB) nodes 128f-h. It will be understood that the number of nodes 128 shown in FIG. 5, as well as the function of these nodes 128, may vary, including consolidating the functionality of one or more of these nodes into fewer nodes, dividing the functionality of one or more nodes 128 into a greater number of nodes 128, adding new nodes with new functionality, and/or eliminating one or more of the nodes 128 shown in FIG. 5. It will also be understood that, in at least one embodiment, embedded network 132 is a Controller Area Network (CAN), although it will be understood that in other embodiments, a different type of embedded network may be utilized, such as, but not limited to, an onboard Ethernet. Still further, in some embodiments, two or more of the nodes 128 may communicate with each other in a wireless fashion.

Main node 128a includes a controller 134 and is coupled to a memory 136. Memory 136 includes the data and programming for carrying out the functions described herein. Memory 136 also includes a patient support apparatus ID 138 that uniquely identifies the patient support apparatus 20. Controller 134 is configured to send ID 138 to server 84, in some embodiments, along with location information so that server 84 is able to tell which particular patient support apparatus 20 is positioned where within a particular healthcare facility. Server 84 may also use the location of the particular patient support apparatus 20 to determine which patient and/or caregiver is assigned to a particular room 58 of the healthcare facility. This determination of the assigned patient or caregiver may be carried out by communication with one or more other servers on network 80 that store data identifying which patients and/or caregivers are assigned to which locations within the healthcare facility.

Patient support apparatus 20 further includes a microphone 140 in communication with main control node 128a and controller 134. Microphone 140 is used by a patient when he or she wishes to speak to a remotely positioned nurse, as will be described in more detail below. Bluetooth node 128b includes a Bluetooth transceiver 122 that is adapted to communicate with the Bluetooth transceiver 106 of wall units 60. Control panel node 128c includes one or more of the control panels 54 of patient support apparatus 20. As shown in FIG. 5, control panel node 128c includes display 52, one or more controls 50, and a display controller 146. Display controller 146 is adapted to control what is displayed on display 52 and to oversee communications between control panel node 128c and the rest of control system 126.

Remote communication node 128d (FIG. 5) includes one or more network transceivers 94 and a communication controller 148. As was noted previously, network transceiver(s) 94 may be WiFi transceivers, Ethernet transceivers, and/or other any other type of transceiver that is capable of allowing patient support apparatus 20 to communicate with network 80 and/or a remote network that is coupled to network 80 (e.g. the Internet 90). Communication controller 148 is adapted to oversee the communications between transceiver 94 and the network 80, as well as to oversee communications between remote communication node 128d and the rest of control system 126.

Motion control node 128e includes a motion controller 150 that is in communication with one or more powered actuators, such as the lift actuators 102 and Fowler actuator 120. Motion controller 150 is responsible for converting motion commands detected on embedded network 132 into motion control signals that are sent to the appropriate actuators 102, 120. Motion controller 150 may also be adapted to report the current position of one or more of the actuators 102, 120 to the other nodes 128 via the embedded network 132. Still further, motion controller 150 may oversee the communications between motion control node 128e and the rest of control system 126.

Each UWB node 128f-h (FIG. 5) includes a UWB transceiver 124 and a UWB controller 142. Each UWB transceiver 124 is positioned at a known location on patient support apparatus 20. This known location information is stored in memory 136 and/or elsewhere, and may be defined with respect to any suitable frame of reference that is fixed with respect to patient support apparatus 20. The known location information may include the spatial relationship between UWB transceivers 124 and/or any other components of patient support apparatus 20. For example, in some embodiments, the known location information includes the spatial relationship not only between UWB transceivers 124, but also the spatial relationships between UWB transceivers 124 and one or more of the following: the head end 38 of patient support apparatus 20, the foot end of patient support apparatus 20, the sides of patient support apparatus 20, the floor, and/or other components and/or landmarks of patient support apparatus 20. In some embodiments, this location information is used to determine the orientation of patient support apparatus 20 with respect to one or more walls 62, wall units 60, another patient support apparatus 20, and/or another object or structure within the healthcare facility.

Each UWB node 128f-h includes a UWB controller 142 in addition to the UWB transceiver. UWB controllers 142 oversee the operation of their respective UWB transceiver 124 and carry out communications with embedded network 132. In some embodiments, UWB controllers 142 correspond to any one or more of the previously mentioned Trimension™ semiconductor chips manufactured by NXP semiconductors (e.g. the NXP SR150, SR100T, SR040, NCJ29D5, and/or the OL23DO chips), or they correspond to any of the microcontrollers incorporated into the Decawave DWM1000, DWM10001C, DWM3000 modules or other modules mentioned above.

Controllers 108, 134, 142 146, 148, and 150, as well as any other controller described herein, may take on a variety of different forms. In the illustrated embodiment, each of these controllers is implemented as a conventional microcontroller. However, these controllers may be modified to use a variety of other types of circuits—either alone or in combination with one or more microcontrollers—such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by the controllers disclosed herein when carrying out the functions described herein, as well as the data necessary for carrying out these functions, are stored in a corresponding memory that is accessible to that particular controller (e.g. memory 136 for controller 134)

Each controller 142 utilizes its associated transceiver 124 to determine the relative position of its associated transceiver 124 with respect to one or more nearby wall units 60 (as well as the position of one or more tags 160 relative to patient support apparatus 20, as discussed more below). If patient support apparatus 20 is positioned within range of a wall unit 60, its UWB transceivers 124 communicate with the UWB transceiver(s) 104 positioned on that wall unit 60, and the transceivers 124 and 104 exchange signals that enable controller 142 to determine the distance between themselves (i.e. distances D1, D2, and D3 in FIG. 5). In some embodiments, UWB transceivers 104, 124 may also be configured to determine an angular relationship between themselves. The distances (and angle information) in at least some embodiments are calculated by one or more of the UWB controllers 142, although it will be understood that these distances and/or angles can be calculated in other locations of control system 126. In other embodiments, UWB transceiver 104 and controller 108 of wall unit 60 may calculate the distances D1-D3 (and angle information) and forward the results of this calculation to patient support apparatus 20 (either via UWB transceiver 104 or BT transceiver 106). In either situation, control system 126 of patient support apparatus 20 is informed of the distances D1, D2, D3, etc. (and, in some embodiments, as noted, the angle information) between transceiver 104 and transceivers 124. To the extent patient support apparatus 20 includes additional UWB transceivers 124 beyond the three shown in FIG. 5, similar distance calculations are made between each of those UWB transceivers 124 and the wall unit's UWB transceiver 104.

Although FIG. 5 (and FIG. 6) only illustrate a single wall unit 60, it will be understood that a typical healthcare facility will include multiple wall units 60 positioned at different locations throughout the facility, including ones positioned within patient rooms and others positioned outside of patient rooms. Typically, at least one linked wall unit 60a will be positioned in each patient room of the healthcare facility, and if the patient room is intended to be occupied by more than one patient (e.g. it includes multiple bays), then additional linked wall units 60a may be included so that each patient support apparatus 20 will have a linked wall unit 60a positioned adjacent to each bay area in the room. Additional wall units 60, such as unlinked wall units 60b, may also be positioned at other locations through the healthcare facility.

In many locations throughout the healthcare facility, transceiver(s) 124 of an individual patient support apparatus 20 will be able to concurrently communicate with multiple wall units 60 because the communication range of the UWB transceivers 104 onboard the wall units 60 will overlap with each other. In such situations, controllers 142 and/or controller 108 may utilize distances between each UWB transceiver 104 and at least one of the UWB transceivers 124 positioned onboard patient support apparatus 20 to determine the location of the patient support apparatus. Such distances, to the extent they are not determined by control system 126 of patient support apparatus 20, are forwarded by the controllers 108 of the respective wall units 60 to patient support apparatus 20 (either via transceivers 104 or 106).

When control system 126 receives multiple distances from multiple wall units 60, it may react in different manners, depending upon the particular embodiment of patient support apparatus 20 that is implemented. In a first embodiment, control system 126 forwards the multiple distances to server 84 via network transceiver 94 and server 84 uses the multiple distances to determine a current location of that patient support apparatus 20 (via one or more conventional methods, such as, but not limited to, triangulation and/or trilateration). In a second embodiment, control system 126 uses the multiple distances to determine a current location of patient support apparatus 20 (via one or more of the same conventional methods) and then forwards the current location to server 84 via network transceiver 94. In another embodiment, control system 126 determines which wall unit 60 is closest to it and uses communications between only that wall unit 60 and its transceivers 124 to determine its location (and thus doesn't rely on communications with multiple wall units 60 to determine its location).

The degree of specificity of the location determined using UWB transceivers 104 and 124 may vary depending upon how close patient support apparatus 20 is to a particular wall unit 60. For example, in some embodiments, control system 126 and/or server 84 conclude that a patient support apparatus 20 is in the same location as a particular wall unit 60 if patient support apparatus 20 is within a threshold location range of that wall unit 60. In such embodiments, the threshold location range may refer to an area having horizontal dimensions of approximately five by ten feet (and a height of approximately five or more feet above the ground) that is positioned directly in front of a linked wall unit 60a, although other dimensions may, of course, be used. For unlinked wall units 60b, the threshold location range may cover a greater area (and volume) of space. If the patient support apparatus 20 is positioned outside of the threshold location range of a particular wall unit 60, control system 126 and/or server 84 may determine an actual coordinate location of patient support apparatus 20 and/or report a more generalized location (e.g. outside of room 402; on the third floor, in corridor X, in the Emergency Department, etc.).

When control system 126 determines multiple distances (whether measured between patient support apparatus 20 and multiple wall units 60 or between patient support apparatus 20 and only a single wall unit 60), control system 126 is adapted to either use those multiple distances to determine the location of patient support apparatus 20, or it is adapted to forward those distances to an off-board entity (e.g. server 84) to use those multiple distances to determine the location of patient support apparatus 20. In either case, control system 126 and/or server 84 may determine the location of patient support apparatus 20 using a coordinate frame of reference in which the position of each wall unit 60 is also known (e.g. the current position of the patient support apparatus is (X, Y, Z) in a frame of reference in which a first wall unit 60 is positioned at (X₁, Y₁, Z₁), a second wall unit is positioned at (X₂, Y₂, Z₂), a third wall unit is positioned at (X₃, Y₃, Z₃), etc.). The current position of the patient support apparatus 20 may then be compared to the one or more threshold location ranges to determine if the patient support apparatus is positioned inside or outside of the threshold location ranges.

The distances D1-D3 (FIG. 5) are determined by an exchange of communication signals between UWB transceivers 104 and 124. This exchange is initiated by an interrogation signal that may be sent by the UWB transceivers 104 of the wall unit 60, and/or it may be sent by the UWB transceivers 124 of the patient support apparatuses 20. The trigger for sending these interrogation signals (from either source) may simply be the passage of a predefined interval of time, in at least some embodiments. That is, in some embodiments, patient support apparatus 20 and/or wall units 60 may be configured to periodically send out an interrogation signal that will be responded to by any UWB transceivers 104 or 124 that are positioned with range of that signal. In those embodiments where patient support apparatuses 20 are configured to send out such an interrogation signal, the time intervals between the interrogation signals may be varied depending upon the location and/or other status of the patient support apparatus 20. For example, in some embodiments, patient support apparatuses 20 may be configured to send out the interrogation signals with longer timer intervals between them when the patient support apparatus is stationary, and to send out the interrogation signals with shorter time intervals between them when the patient support apparatus 20 is in motion. Indeed, in some embodiments, after patient support apparatus 20 has ceased moving, control system 126 may be configured to cease sending out such interrogation signals until it once again starts moving. In any of the aforementioned embodiments, motion of the patient support apparatus 20 may be detected in any suitable manner, such as by including one or more motion sensors on the patient support apparatus 20 (e.g. one or more accelerometers), and/or by monitoring the values of the repetitive distance measurements and looking for changes indicative of movement.

The measured distances (and/or angular information between wall units 60 and patient support apparatuses 20) that are generated from the communications between UWB transceivers 104, 124 may utilize Angle of Arrival (AoA) information, Time of Flight (TOF) information, Channel State Information, Time Difference of Arrival (TDoA) information, Two-Way Ranging (TWR) ranging information, and/or other information. In some embodiments, each transceiver 104, 124 includes an array of antennas that are used to generate distance and/or angular information with respect to the transceiver 104, 124 in which it is in communication. In some embodiments, one or more of the UWB controllers 142 determine the relative position of transceivers 104, 124 without utilizing main controller 134 and/or controller 108. In other embodiments, controllers 108 and/or 134 may work in conjunction with the controllers 142 to determine the relative locations of transceivers 104 and 124 to each other.

Patient support apparatus 20 also includes, in at least some embodiments, a microphone 140 (FIG. 5) that is used to detect the voice of the patient when the patient wants to speak to a remotely positioned nurse. The patient's voice is converted to audio signals by microphone 140 and controller 134 is adapted to forward these audio signals to an adjacent communications outlet 64 positioned in wall 62 (FIG. 4). When a cable 66 is coupled between patient support apparatus 20 and outlet 64, controller 134 forwards these audio signals to outlet 64 via the cable. When no such cable 66 extends between patient support apparatus 20 and outlet 64, controller 134 wirelessly forwards these audio signals to headwall unit 60 (using transceiver 122, or in some embodiments, one or more of transceivers 124)) and controller 108 of headwall unit 60 forwards these audio signals to outlet 64. As was noted, outlet 64 is in electrical communication with a conventional nurse call system 70 that is adapted to route the audio signals to the correct nurse's station 76, and/or other location. In some embodiments, microphone 140 acts as both a microphone and a speaker. In other embodiments, a separate speaker may be included in order to communicate the voice signals received from the remotely positioned nurse. In some embodiments, the audio communication between patient support apparatus 20 and communications outlet 64 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

After the installation of wall units 60a and 60b in a particular healthcare facility, the location of each wall unit 60 within that facility is recorded. In some embodiments, the coordinates of the locations of wall units 60 are recorded in a common frame of reference (or converted to a common frame of reference after recordation). Such coordinates may be three dimensional (i.e. include a height components), or they may be two dimensional (no height component). In other embodiments, a more generalized location of one or more wall units 60 is determined, rather than the precise coordinates of the wall units 60. In still other embodiments, the locations of one or more wall units 60 are determined both generally and more precisely. The generalized location of the wall units 60 may include an indication of the room, bay, area, hallway, portion of a hallway, wing, maintenance area, etc. that the wall unit 60 is positioned in. The specific location of the wall units 60, as noted, may include an X, Y, and Z coordinate within a common frame of reference.

In some embodiments, the location of each wall unit 60 (whether specific and/or general) is stored in a memory within that particular wall unit 60 and shared with the devices it communicates with (e.g. patient support apparatuses 20). In some embodiments, the location of each wall unit 60 is stored within memory 136 (FIG. 5) of each patient support apparatus 20. Still further, in some embodiments, the location of each wall unit 60 is stored within a memory accessible to server 84. Alternatively, or additionally, the location of each wall unit 60 may be stored in two or more of the aforementioned locations.

It will be appreciated that patient support apparatuses 20 are configured to communicate with wall units 60 regardless of the orientation of the patient support apparatus 20. That is, the UWB transceivers 104 and 124 are radio frequency transceivers that do not rely on line of sight communication, unlike the IR transceiver 118 (if present). Thus, the patient support apparatuses 20 do not have to be pointed in any particular direction with respect to the wall units in order for transceivers 104 and 124 to communicate. This differs from some prior art systems that use IR communication between the patient support apparatuses 20 and the wall units and that require the IR transceiver onboard the patient support apparatus to be aimed toward the wall unit in order for communication to be established.

It will also be understood that, although units 60 are referred to herein as “wall units” 60, such units do not necessarily have to be positioned on walls. That is, wall units 60 can be positioned on columns, ceilings, or any other fixed structures within the healthcare facility. It will therefore be understood that the term “wall” as used herein is not exclusive of ceilings, columns, or other fixed architectural structures.

Although the distances D1-D3 (FIG. 5) between each UWB transceiver 124 on patient support apparatus 20 and the UWB transceiver 104 on wall unit 60 may be determined in a variety of different manners, in at least one embodiment, control system 126 is configured to determine these distances by using a time difference or arrival (TDoA) method. In this method, transceiver 104 of wall unit 60 emits a radio signal, or ping, that is detected by each of the UWB transceivers 124 onboard patient support apparatus 20. The time at which each UWB transceiver 124 detects this ping is recorded. By comparing the differences in the time of arrival between each of the UWB transceivers 124, the distances between D1-D3 can be determined. Further, from distances D1-D3, control system 126 is configured to determine the relative three dimensional position of patient support apparatus 20 with respect to the wall unit 60. In some embodiments, this time difference of arrival method may utilize additional information, such as angular information determined from the communications between transceivers 124 and transceiver 104. Alternatively, or additionally, more than three UWB transceivers 124 may be positioned onboard patient support apparatus 20 in order to determine additional distances, which may allow more precise position determinations to be made of patient support apparatus 20.

In some embodiments, controllers 142 are configured to synchronize a time measurement with each other so that they can accurately determine a time difference of arrival from the ping sent by UWB transceiver 104. In other words, in order for control system 126 to accurately determine the differences between the arrival times of the ping at the different UWB transceivers 124, each UWB node 128f-h must have their measurement of time synchronized. In at least one embodiment, nodes 128f-g synchronize their time measurements using communications over the embedded network 132, which, as noted, may be a CAN network.

Each UWB node 128f-g synchronizes its measurement of time with the other nodes 128f-g by sending one or more synchronization messages to the other nodes 128f-g. In at least one embodiment, these synch messages are sent with a priority level that is higher than other communications that take place over the embedded network 132. Thus, for example, if network 132 is a CAN network, the synchronization messages are sent with a CAN identifier that has a lower number, and thus a higher priority, than all of the other types of messages that are sent over the CAN network 132. In this manner, if another node on the embedded network 132 attempts to send a message at the same time that one of the synchronization messages is being sent, the synchronization message will be sent and the message from the other node will not get sent. The synchronization messages may contain any suitable and/or conventional synchronization data that is appropriate for ensuring that nodes 128f-h have their clocks, or measurements of time, synchronized with each other.

In some embodiments, the synchronization messages sent over embedded network 132 are sent in a raw CAN format, while the non-synchronization messages sent over the embedded network 132 are sent in a non-raw CAN format (e.g. CANOpen, the J1939 protocol, etc.). These non-synchronization messages include any type of message that is sent by any one of the nodes 128 that does not effectuate synchronization of the nodes 128e-g. Thus, for example, control panel node 128c may be configured to send out a motion control command to motion control node 128e via network 132 in response to a user activating a motion control 50 that is part of a control panel 54. This motion control command may instruct motion control node 128e, for example, to activate a Fowler actuator 102 to raise Fowler section 44 on patient support apparatus 20. This motion control command is sent with a CAN ID that is higher than that used for the synchronization messages. In this manner, if the motion control command is sent at the same time as a synchronization message, the synchronization message will get through and the motion control command will need to be sent at a subsequent time.

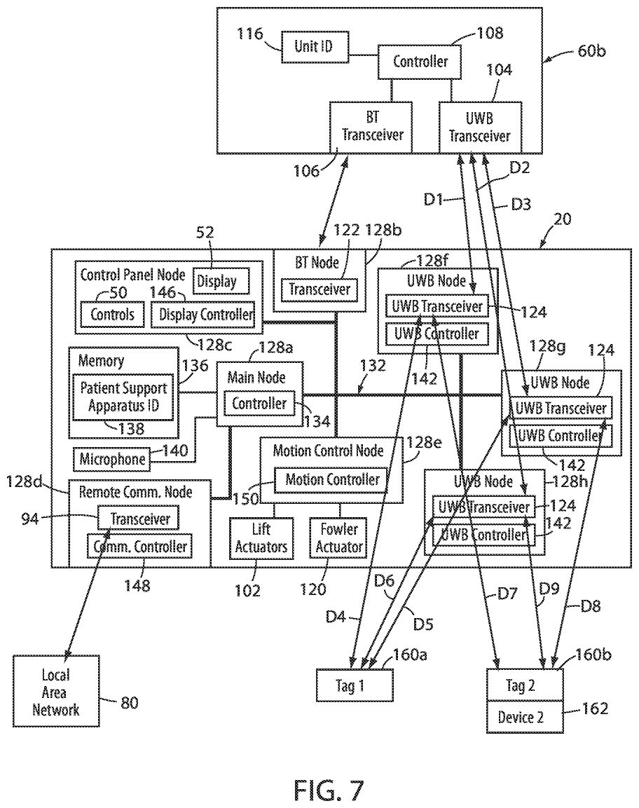

FIGS. 6 and 7 illustrate an unlinked wall unit 60b that functions to provide location information to one or more patient support apparatuses 20 in the same manner as has been described herein (and as is described further below). Unlinked wall units 60b differ from linked wall units 60a in that unlinked wall units 60b are not communicatively coupled to a communications outlet 64. As a result, unlinked wall units 60b can be positioned at any fixed location within the healthcare facility, rather than only at locations that are next to a communications outlet 64. Because such communication outlets 64 are typically only placed along the headwalls within patient rooms, linked wall units 60a are typically only positioned on the headwalls of patient rooms. Unlinked wall units 60b, however, are able to be placed in hallways, in elevators, in maintenance areas, parking garages, and/or at any other desired locations where patient support apparatuses 20 are expected to travel and where location information regarding the patient support apparatuses 20 is desired.

FIG. 6 illustrates a wall unit 60b positioned in a hallway. FIG. 7 illustrates the internal components of wall unit 60b. As can be seen in FIG. 7, wall unit 60b does not include a number of components that may be found in linked wall units 60a, such as configuration circuitry 110, television controller 112, headwall interface 114, and/or IR transceiver 118. Instead, wall unit 60b includes a controller 108, Bluetooth transceiver 106, UWB transceiver 104, and a unit ID 116. These four components work in the same manner as the corresponding components of linked wall units 60a. That is, UWB transceiver 104 is used in conjunction with transceivers 124 to determine the distances D1-D3 (and/or to determine an angular relationship between wall unit 60b and the transceivers 124). Controller 108 sends unit ID 116 to patient support apparatus 20, as well as any location and/or angular calculations that it makes in response to the communications between UWB transceiver 104 and UWB transceiver(s) 124. BT transceiver 106 may be used for any high bandwidth communications between wall unit 60b and patient support apparatus 20 that require, or are desirably carried out with, a communication channel having greater bandwidth than the communication channel between UWB transceivers 104 and 124. In some embodiments, BT transceiver 106 may be omitted entirely from unlinked wall units 60b.

Further details regarding the manner(s) in which patient support apparatus 20 may interact with one or more wall units 60 in order to determine their location, and in some cases, their orientation, are described in commonly assigned U.S. patent application Ser. No. 63/245,245 filed Sep. 17, 2021, by inventors Kirby Neihouser et al., and entitled SYSTEM FOR LOCATING PATIENT SUPPORT APPARATUSES, the complete disclosure of which is incorporated herein by reference. Wall units 60a and 60b, as well as patient support apparatuses 20, may include any of the same functionality and/or components as the wall units 60a and 60b and patient support apparatuses 20 that are described in the aforementioned '245 patent application.

In addition to being used to determine the location of patient support apparatus 20 within a healthcare facility, transceivers 124 are also used to determine the location of one or more tags 160 (FIGS. 4-8) with respect to patient support apparatus 20. For purposes of the following written description, tags 160 that are attached to a patient 164 (see FIGS. 4 & 6) will be referred to as tags 160a herein, while tags 160 that are attached to a device 162 will be referred to herein as tags 160b (FIGS. 5, 7, & 8). Both tags 160a and 160b may be of the same construction with their only difference being the entity to which they are attached. Alternatively, tags 160a and 160b may be constructed in different manners. For purposes of the following written description, reference to “tag 160” or “tags 160” will be understood to apply to both tags 160a and 160b.

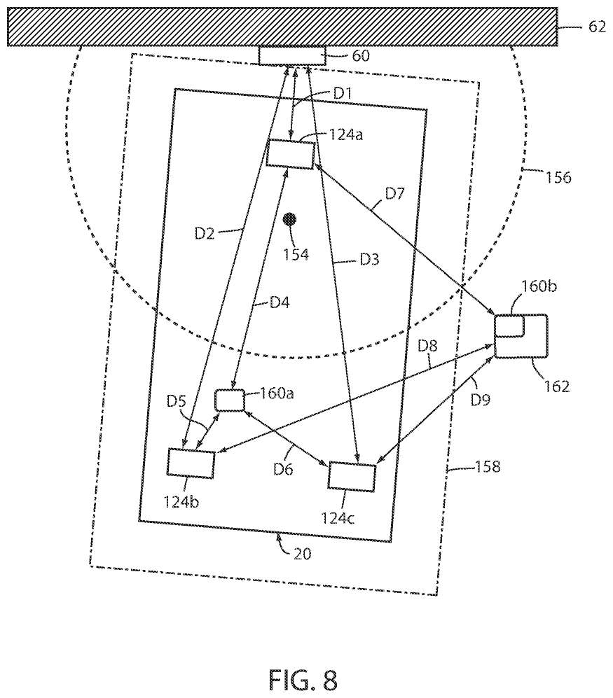

Tags 160 are adapted to use ultra-wideband (UWB) communication to communicate with UWB transceivers 124 positioned onboard patient support apparatus 20. Tags 160 may also be adapted to communicate with one or more of the UWB transceivers 104 positioned inside of wall units 60. Control system 126 uses the UWB communications between transceivers 124 and the tag(s) 160 to determine the distance between each transceiver 124 and each of the tag(s) 160. For example, as shown in FIG. 8, control system 126 uses UWB communications between transceivers 124a-c and tag 160a to determine distances D4, D5, and D6 between transceivers 124a-c, respectively, and tag 160a. Similarly, control system 126 uses UWB communications between transceivers 124a-c and tag 160b to determine distances D7, D8, and D9 between transceivers 124a-c, respectively, and tag 160b. In some embodiments, control system 126 also gathers angular information from each of the transceivers 124a-c regarding the angular relationship of each of those transceivers 124a-c to each of the tags 160. Whether such angular information is gathered or not, control system 126 uses the distance measurements to determine the three-dimensional position of each of the tags 160 that are within communication range of UWB transceivers 124 (or that are within a maximum threshold distance of these transceivers 124). In some embodiments, patient support apparatus 20 may include more than the three UWB transceivers 124 shown in FIG. 8, which may give control system 126 more precise information about the current location of each tag 160. Regardless of the number of UWB transceivers 124, control system 126 may use conventional trilateration, triangulation, and/or other algorithms for determining the three dimensional position of the tags 160.

The three dimensional position of the tags 160 is determined within a coordinate frame of reference that is local to the patient support apparatus 20, and that may or may not be correlated to the frame of reference that is used to determine the location of patient support apparatus 20 within the healthcare facility. That is, control system 126 uses a local frame of reference for determining the position of tags 160 that is based off of a fixed landmark on patient support apparatus 20, and the positions of each of the UWB transceivers 124 within that local frame of reference is known to control system 126 (e.g. stored in memory 136). Because this local frame of reference is independent of the frame of reference used for determining locations within the entire healthcare facility, the three dimensional position of a tag 160 that does not move with respect to patient support apparatus 20 will retain the same three dimensional position within the local frame of reference, even if the patient support apparatus 20 is moved to different locations within the healthcare facility.

In some embodiments, control system 126 uses the position information from one or more patient worn-tags 160a to carry out one or more patient monitoring functions associated with patient monitoring control 50a (FIG. 2). Such patient-monitoring functions are described in greater detail in commonly assigned U.S. patent application Ser. No. 63/245,279, filed Sep. 17, 2021, by inventors Jerry Trepanier et al. and entitled PATIENT SUPPORT APPARATUSES WITH PATIENT MONITORING, the complete disclosure of which is incorporated herein by reference. Control system 126 also uses the position information from the one or more device-attached tags 160b to carry out association and/or data gathering functions regarding the devices to which the tags 160b are attached.

Tags 160 may be constructed to include their own internal UWB transceiver that is similar, if not the same as, the UWB transceivers 124 and/or 104. In some embodiments, tags 160 include any of the Trimension™ ultra-wideband modules available from NXP Semiconductors of Austin, Texas. These modules include, but are not limited to, the Trimension™ UWB modules ASMOP1BO0N1, ASMOP1CO0R1, and/or the ASMOP1CO0A1, and that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement tags 160. Further details regarding one manner in which tags 160 may be physically constructed are provided in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference. Still other physical constructions of tags 160 may be utilized.

Patient tags 160a may be constructed to attach to a patient 164 in any suitable manner. In some embodiments, patient tags 160a are incorporated into a wristband, ankle band, or some other type of band that is wrapped around a portion of the patient's anatomy. Alternatively, patient tags 160a may include an adhesive that is used to adhere the tag 160a to the patient's skin and/or clothing. As another alternative, tags 160a may be sewn into, or otherwise integrated into, one or more of the items of clothing worn by the patient. Still other manners of securing tags 160a to the patient may be utilized.

In addition to determining the position of one or more tags 160 with respect to a local frame of reference (tied to patient support apparatus 20), control system 126 uses UWB transceivers 124 to determine the location of patient support apparatus 20 within the healthcare facility. As was described above, this location within the healthcare facility may utilize a general frame of reference that is fixed with respect to the healthcare facility, and thus independent of the local frame of reference of patient support apparatus 20. Thus, in the example shown in FIG. 8, control system 126, in addition to determining distances D4-D9, also determines distances D1-D3, which are the distances between each transceiver 124 and the adjacent wall unit 60. From these distances D1-D3, as well as the wall unit ID 116 received from wall unit 60 (and, in some embodiments, additional information received from wall unit 60), control system 126 is able to determine the overall location of patient support apparatus 20 within the healthcare facility.

More particularly, control system 126 may determine the location of patient support apparatus 20 within the healthcare facility by comparing a current position of a reference point 154 (FIG. 8) onboard patient support apparatus 20 to a threshold range 156 defined with respect to an adjacent wall unit 60. The threshold range 156 defines a volume of space adjacent to, and fixed with respect to, the wall unit 60. In some embodiments, this threshold range 156 is the same for each wall unit 60, while in other embodiments, it may vary from unit 60 to unit 60. If the reference point 154 (whose position relative to transceivers 124 is known and stored in memory 136) is positioned inside of the threshold range 156 (as shown in FIG. 8), then control system 126 determines that patient support apparatus 20 should associate itself with the adjacent wall unit 60. If the reference point is outside of the threshold range 156 (not shown in FIG. 8), then control system 126 determines that patient support apparatus 20 should not associate with the adjacent wall unit 60. Further details regarding the meaning, function, and ramifications of this association process are found in the aforementioned commonly assigned U.S. patent application Ser. No. 63/245,245, the complete disclosure of which has already been incorporated herein by reference.

Control system 126, in some embodiments, is also configured to monitor the location of one or more tags 160b that are attached to devices 162, rather than to patient 164. In some embodiments, control system 126 is configured to automatically determine if a tag 160b that is coupled to a device is positioned within an association threshold 158 of patient support apparatus 20 (FIG. 8). If the tag 160b is positioned within the association threshold 158, control system 126 automatically concludes that the tag 160b is attached to a device 162 that is to be associated with that particular patient support apparatus 20, as well as the patient assigned to that particular patient support apparatus 20. Thus, for example, if a tag 160b attached to a ventilator is positioned within association threshold 158, control system 126 concludes that the ventilator is being used with the patient onboard patient support apparatus 20. Control system 126 may then automatically take one or more additional actions in response to this determination. These additional actions include, but are not limited to, informing server 84 of the determination that the tag 160b (and its associated device) should be associated with patient support apparatus 20 (and the patient assigned thereto), performing an authentication analysis of device 162 to ensure it is an authorized device for communicating with patient support apparatus 20, forwarding data from tag 160b and/or the attached device to server 84, and/or other actions.

Association threshold 158 defines a three-dimensional volume of space (FIG. 8) that is fixed with respect to patient support apparatus 20. Generally speaking, association threshold 158 encompasses the volume occupied by patient support apparatus 20, as well as a certain amount of space around the perimeter of patient support apparatus 20. This perimeter space allows for the automatic association of one or devices that are customarily positioned adjacent to patient support apparatus 20, such as, but not limited to, a portable IV pole. In some embodiments, control system 126 forwards the location of tag 160b to server 84 and server 84 determines whether the tag 160b (and associated device) should be associated with that particular patient support apparatus 20.

When patient support apparatus 20 and/or server 84 associate a tagged device with a particular patient support apparatus 20, control system 126 and/or server 84 are configured to inform medical personnel (via electronic devices 96) that the tagged device is associated with a particular patient support apparatus 20 and/or with a particular patient assigned to that patient support apparatus 20. In this manner, data from the device can be correlated with a particular patient. Indeed, in some embodiments, patient support apparatus 20 is configured to automatically establish communication with the tagged device (via transceiver 124 and/or Bluetooth transceiver 122) and to forward data from that device to server 84, which in turn may automatically forward the data to the corresponding patient's electronic medical record. Because patient support apparatus 20 automatically determines that the device is positioned within the volume of space defined by association threshold 158, it is not necessary for a caregiver to take any manual steps to ensure that data from the tagged device is forwarded to the proper corresponding patient's electronic medical record because patient support apparatus 20, along with server 84, automatically determine the correct patient associated with that tagged device. Further details regarding at least one manner in which this automatic patient association may be made are found in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference.

It will be understood that a tag 160b may be attached to a wide variety of types of devices. Such devices include, but are not limited to, another patient support apparatus 20, an infusion pump, a vital sign sensor, an exercise device, a heel care boot, an IV stand and/or pole, a ventilator, a DVT pumps, a patient monitor (e.g. a saturated oxygen (SpO₂) monitor, an EKG monitor, a vital sign monitor, etc.), a patient positioning devices (e.g. a wedge, turning device, pump), an ambient sensor (e.g. air temperature, air flow, light, humidity, pressure, altitude, sound/noise), a mattress 42, a portable exit detection sensor, an attachable nurse call device, an incontinence pad or one or more sensors adapted to detect patient incontinence, a Holter device adapted to monitor and record a patient's heart signals, a patient ID tag or bracelet worn by the patient that identifies the patient, a caregiver tag or ID bracelet worn by a caregiver that identifies the caregiver, a patient temperature management device (or associated device, such as a one or more hoses, thermal wraps, etc.), one or more mobility assistance devices that a patient may be expected to use, and/or any other device that may be used when caring for a patient.

In at least one embodiment, patient support apparatus 20 may be configured to detect the position of an exercise device (and/or movement of the exercise device) by incorporating a tag 160b into the exercise device. One example of this is disclosed in commonly assigned U.S. patent application Ser. No. 63/161,175 filed Mar. 15, 2021, by inventors Krishna Bhimavarapu et al. and entitled EXERCISE DEVICE AND PATIENT SUPPORT APPARATUS, the complete disclosure of which is incorporated herein by reference.

In those embodiments where devices 162 include an infusion pump, patient support apparatus 20 and patient support apparatus server 84 may be configured to carry out any of the functions associated with the infusion pump that are described in commonly assigned U.S. patent application Ser. No. 63/349,369 filed Jun. 6, 2022, by inventors Krishna Bhimavarapu et al. and entitled COMMUNICATION SYSTEM FOR PATIENT SUPPORT APPARATUSES, the complete disclosure of which is incorporated herein by reference. In those embodiments where devices 162 include a portable exit detection sensor, another patient support apparatus, and/or an attachable nurse call device, patient support apparatus 20 and patient support apparatus server 84 may be configured to carry out any of the functions associated with the portable exit detection sensors, nurse call devices, and secondary patient support apparatuses disclosed in commonly assigned U.S. patent application Ser. No. 63/352,061 filed Jun. 14, 2022, by inventors Jerald Trepanier et al. and entitled COMMUNICATION SYSTEM FOR PATIENT SUPPORT APPARATUSES, the complete disclosure of which is incorporated herein by reference. In those embodiments where devices 162 include one or more vital sign sensors, patient support apparatus 20 and patient support apparatus server 84 may be configured to carry out any of the functions associated with the vital sign sensors and/or display devices disclosed in commonly assigned U.S. patent application Ser. No. 63/306,279 filed Feb. 3, 2022, by inventors Madhu Thota et al. and entitled COMMUNICATION SYSTEM FOR PATIENT SUPPORT APPARATUSES, the complete disclosure of which is incorporated herein by reference. In those embodiments where devices 162 include one or more patient temperature management devices, patient support apparatus 20 and patient support apparatus server 84 may be configured to carry out any of the functions associated with the patient temperature management devices disclosed in commonly assigned U.S. patent application Ser. No. 63/314,221 filed Feb. 25, 2022, by inventors Jerry Trepanier et al. and entitled COMMUNICATION SYSTEM FOR PATIENT SUPPORT APPARATUSES AND TEMPERATURE MANAGEMENT DEVICES, the complete disclosure of which is incorporated herein by reference.

In some embodiments, the transceivers 104, 124, nodes 128f-h, and/or tags 160 may operate in the same manner as, and include any of the same functions as, the anchors and pseudo-anchors disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference.

In some embodiments, wall units 60 may also be utilized to determine the location of any of tags 160a and/or 160b, such as is disclosed in commonly assigned U.S. patent application Ser. No. 63/132,514 filed Dec. 31, 2020, by inventors Alexander Bodurka et al. and entitled PATIENT SUPPORT APPARATUS AND MEDICAL DEVICE NETWORKS, and in commonly assigned U.S. patent application Ser. No. 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosures of both of which are incorporated herein by reference.

As was mentioned previously, in some embodiments, tags 160a and 160b include different IDs, or different types of IDs, so that control system 126 is able to distinguish between tags 160a that are attached to a patient's body and tags 160b that are attached to devices. In some embodiments, the IDs may tell control system 126 additional information, such as the location of a particular tag 160a on the patient's body, the type of device to which a tag 160b is attached (e.g. a heel care boot, a vital signs monitor, a patient monitor, an IV stand, a therapy device, etc.), and/or other information. The ID of the tag 160b is transmitted to one or more of the transceivers 124 onboard patient support apparatus 20, and patient support apparatus 20 is configured to then forward the ID to server 84 and/or one or more electronic devices 96. The recipient of the ID has access to a data table that correlates the ID to a specific type of device, and the recipient can then share the fact that a particular type of device 162 is being used with a patient on a particular patient support apparatus 20. This sharing may take place via one or more of the electronic devices 96, thereby enabling the electronic devices 96 to display the type of device(s) being used with a particular patient. Data from the device may also be displayed on the same electronic device 96, thereby giving the viewer real time information about the devices being used with a particular patient support apparatus.

In any of the embodiments disclosed herein, server 84 may be configured to additionally execute a caregiver assistance software application of the type described in the following commonly assigned patent applications: U.S. patent application Ser. No. 62/826,097, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. That is, server 84 may be configured to share with one or more electronic devices any of the information shared with the electronic devices disclosed in these aforementioned patent applications. Thus, for example, server 84 may be configured to not only share the location of patient support apparatuses 20 (and any devices that may be associated with them) with electronic devices 96, but it may also forward data received from any of the wall units 60 and/or tags 160 (or coupled devices 162) 136 to the electronic devices 96, thereby letting the caregivers associated with these devices know this data. Alternatively, or additionally, patient support apparatus server 84 may forward patient support apparatus status data (e.g. current siderail position, bed exit status, brake status, height status, scale data, etc.) and/or caregiver rounding information (e.g. when the last rounding was performed for a particular patient, when the next rounds are due, etc.), and/or position data regarding any of the tags 160a, 160b that are positioned within range of UWB transceivers 124 to one or more electronic devices 96, thereby providing the caregivers associated with the devices 96 a consolidated portal (e.g. a single software application) for sharing this various information.

In some embodiments, wall units 60a and/or 60b may be configured to send a signal to patient support apparatus 20 (via UWB transceiver 104 and/or via BT transceiver 106) indicating what type of wall unit it is (e.g. whether it is a linked wall unit 60a or an unlinked wall unit 60b). Control system 126 of patient support apparatus 20 uses this information to determine whether or not to forward audio signals from microphone 140 to the wall unit 60 via BT transceiver 122 or UWB transceiver 124. More specifically, if the wall unit is a linked wall unit 60a, control system 126 forwards the audio signals (and linked wall unit 60a then forwards the audio signals to the communications outlet 64). If the wall unit is an unlinked wall unit 60b, control system 126 does not forward the audio signals (but instead forwards them to a nurse call cable port that receives a nurse call cable 66). The audio signals are generated from the patient's voice when he or she is talking to a remotely positioned caregiver via the healthcare facility's nurse call system.

It will also be understood that, although patient support apparatus 20 has been primarily described as having three UWB transceivers 134a-c (e.g. FIG. 8), patient support apparatus 20 may have different numbers of UWB transceivers in different embodiments, including embodiments with only a single transceiver 124, two UWB transceivers 124, four UWB transceivers 124, or more than four UWB transceivers 124.

Additionally, in some embodiments, patient support apparatus 20 may be adapted to display on one or more of its own displays—which may be included within any of control panels 54—its location as determined by its communication with one or more wall units 60. Additionally, or alternatively, patient support apparatus 20 may also be configured to display on display 52 the current position of any or all of the tags 160 whose current position it has determined.

It will also be understood that, in some embodiments, patient support apparatus 20 may include one or more UWB transceivers 124 that are positioned on movable components of patient support apparatus 20, such as, but not limited to, litter frame 28 and/or siderails 36. In such embodiments, control system 126 is apprised of the current location of each moveable transceiver 124 by way of one or more sensors that measure the current location of the one or moveable components. Control system 126 then uses this current location information when it communicates with one or more wall units 60 and/or with one or more of the tags 160.

It will be understood that, although the majority of the above-disclosure has discussed the use of transceivers 104 and 124 for determining the distances between themselves and/or one or more tags 160, transceivers 104 and/or 124 may be additionally and/or alternatively used for determining angular information between themselves and/or one or more tags. Thus, in some embodiments, transceivers 124 and the transceivers within tags 160 may be used to not only determine how far each transceiver 124 is positioned from the tag 160, but also the angular relationship of each transceiver 124 relative to the nearby tag 160.

It will also be understood that the operations undertaken by control system 126, as described herein, may be undertaken by any one or more of the controllers that are part of control system 126. Thus, for example, when control system 126 determines the position of patient support apparatus 20 relative to a wall unit 60, the actual calculation of this position may be performed by any one or more of controllers 142, and/or by main controller 134, or by still other controllers that are part of control system 126. Thus, it will be understood that the references herein to action undertaken by control system 126 generically refer to actions that may be taken by any one or more of the controllers described herein.

After control system 126 determines the position of patient support apparatus 20 relative to a nearby wall unit 60 and/or after it determines the relative position of a tag 160 relative to patient support apparatus 20, control system 126 is configured, in some embodiments, to carry out an authentication analysis to determine if the device (wall unit 60, tag 160, and/or device 162 coupled to the tag 160b) is an authentic device. That is, after control system 126 receives identification data from the device it then carries out an authentication analysis on the identification data to confirm that the device is an authentic device of the type indicated in the identification data. In some embodiments, the identification data includes an identity of the manufacturer of the device, as well as additional information discussed below, and control system 126 uses the authentication analysis to confirm that the device actually was manufactured by that particular manufacturer and/or that it correctly corresponds to being what it purports to be.

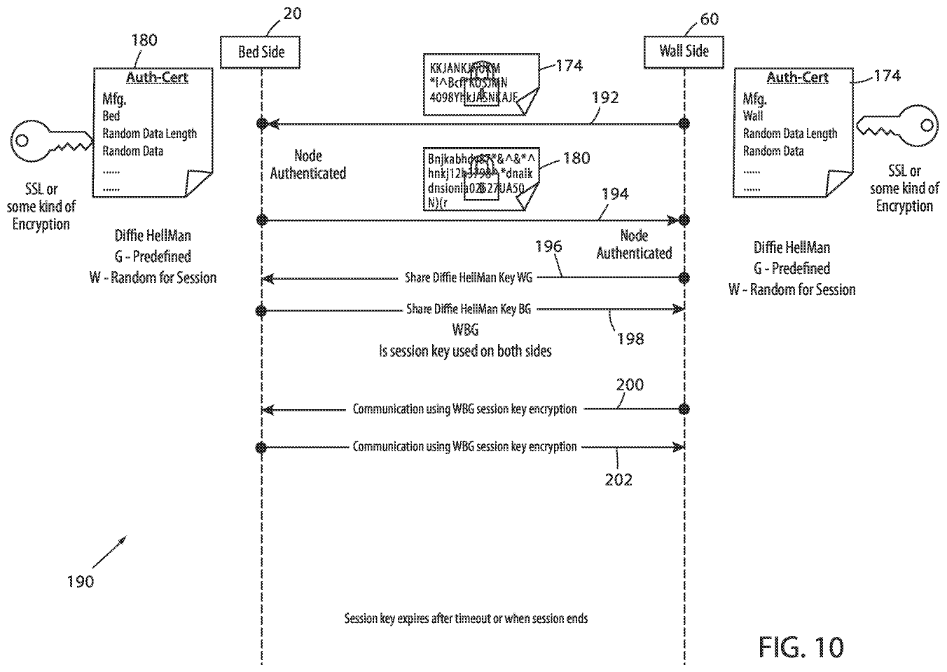

FIGS. 9 and 10 illustrate two different examples of an authentication analysis that may be performed by control system 126 after it establishes communication with a unit 60 or tag 160 (or device 162). In some embodiments, control system 126 is configured to carry out such an authentication analysis only after it first determines that the device (60, 160, 162) is positioned within a threshold distance of patient support apparatus 20. For example, in some embodiments, control system 126 does not attempt to authenticate a wall unit 60 until reference point 154 is positioned within threshold range 156 (FIG. 8). Similarly, in some embodiments, control system 126 does not attempt to authenticate a tag 160 and/or a device 162 unless the tag 160 is positioned within the association threshold 158. In such embodiments, control system 126 only carries out the authentication analysis on a wall unit 60 when reference point 154 is positioned inside threshold range 156, and only carries out the authentication analysis on a tag 160 (or tagged device 162) when the tag or device is positioned inside of association threshold 158.

FIG. 9 illustrates a first authentication algorithm 170 that may be used by control system 126 when performing an authentication analysis on an adjacent wall unit 60 (i.e. a wall unit 60 whose threshold range 156 encompasses reference point 154 of patient support apparatus 20). In some embodiments, the portion of authentication algorithm 170 that is carried out between patient support apparatus 20 and wall unit 60 may be carried out by sending communications over Bluetooth transceivers 106 and 122. In other embodiments, this portion of authentication algorithm 170 may be carried out using UWB transceiver 104 and one or more of the UWB transceivers 124 onboard patient support apparatus 20. In still other embodiments, this portion of authentication algorithm 170 may be carried out using a mix of the Bluetooth and UWB transceivers. The portion of algorithm 170 that is carried out between patient support apparatus 20 and server 84 may be carried out using network transceiver 94.

Authentication algorithm 170 (FIG. 9) begins at an initial step 172 when the wall unit 60 sends a wall unit certificate 174 to patient support apparatus 20. The contents of wall unit certificate 174 may vary from embodiment to embodiment. In one embodiment, wall unit certificate 174 includes a product name of the wall unit 60, a company name of the manufacturer of the wall unit 60, a set of key derivation data, and a set of random data of a random length. The random data may be different each time wall unit 60 establishes, or attempts to establish, a communications session with a patient support apparatus. It will be understood that wall unit certificate 174 may include additional, and/or alternative data. The transmission of the wall unit certificate 174 at step 172 may be carried out by encrypting the message(s) containing the certificate 174. Such encryption may utilize any conventional encryption technology, such as, but not limited to, that based on password key derivation (HMAC (hash-based message authentication code), PBKDF (password-based key derivation function)/PBKDF2), or public key cryptography (RSA (Rivest-Shamir-Adleman)/DSS (Digital Subscriber Standard)).

When patient support apparatus 20 receives wall unit certificate 174, it sends the certificate 174 to server 84 at step 176. In some embodiments, patient support apparatus 20 sends its own patient support apparatus certificate 180 along with the wall unit certificate 174. In such embodiments, server 84 analyzes the patient support apparatus certificate 180 to determine if the message it received at step 176 was sent from an authentic patient support apparatus 20 or not. If not, server 84 does not respond to the message sent at step 176. If server 84 determines from the patient support apparatus certificate 180 that the message sent at step 176 was sent from an authentic patient support apparatus 20, it then proceeds to analyze the wall unit certificate 174.

Server 84 includes a database of information about all of the types of authentic devices that patient support apparatus 20 is authorized to communicate with, and this database includes sufficient information for server 84 to determine the authenticity, or lack of authenticity, of the wall units certificate 174. In some embodiments, the certificate 174 may include information about the date of manufacture of the wall unit 60, the place of manufacture, and/or other information that isn't publicly available regarding wall unit 60. Such information is also stored in server 84, and server 84 uses this information, as well as the other data in certificate 174 described above to determine the authenticity of the certificate 174.

At step 178 (FIG. 9), server 84 sends the results of its authentication analysis back to patient support apparatus 20. If server 84 has determined that wall unit 60 is not an authentic wall unit 60, patient support apparatus 20 does not establish a communication session with wall unit 60, and algorithm 170 terminates until another wall unit 60, or other devices or tag 160, is detected within the threshold range 156 or 158. If server 84 has determined that wall unit 60 is an authentic wall unit 60, server 84 includes within the message sent at step 178 the public key of the wall unit. When wall unit 60 is determined to be authentic, control system 126 of patient support apparatus 20 sends an encrypted message to wall unit 60 at step 182. The encrypted message of step 182 is sent using the public key of the wall unit 60 that server 84 informed patient support apparatus 20 of in the message sent at step 178. The message of step 182 may include the public key of patient support apparatus 20. Wall unit 60 decrypts this message and uses the public key of patient support apparatus 20 for future communications with patient support apparatus 20 at step(s) 184. Similarly, patient support apparatus 20 uses the public key of wall unit 60 for any further communications with patient support apparatus at step(s) 186.

Steps 184 and 186 may be repeated for as long as an individual communication session between patient support apparatus 20 and wall unit 60 continues. In some embodiments, the communication session with a wall unit 60 continues until reference point 154 moves outside of threshold range 156 (or the communication is otherwise interrupted or fails). A new communication session is then established when reference point 154 of patient support apparatus 20 moves within threshold range 156 (either of the same wall unit 60, or a different wall unit 60).

FIG. 10 illustrates a second authentication algorithm 190 that may be used by control system 126 when performing an authentication analysis on an adjacent wall unit 60 (i.e. a wall unit 60 whose threshold range 156 encompasses reference point 154 of patient support apparatus 20). Authentication algorithm 190 may be used in lieu of authentication algorithm 170 so that, in some embodiments, patient support apparatus 20 uses algorithm 170, while in other embodiments, patient support apparatus 20 uses algorithm 190.

As with algorithm 170, in some embodiments, the portion of authentication algorithm 190 that is carried out between patient support apparatus 20 and wall unit 60 may be carried out by sending communications over Bluetooth transceivers 106 and 122. In other embodiments, this portion of authentication algorithm 190 may be carried out using UWB transceiver 104 and one or more of the UWB transceivers 124 onboard patient support apparatus 20. In still other embodiments, this portion of authentication algorithm 190 may be carried out using a mix of the Bluetooth and UWB transceivers.

Authentication algorithm 190 (FIG. 10) begins at an initial step 192 when the wall unit 60 sends a wall unit certificate 174 to patient support apparatus 20. The contents of wall unit certificate 174 may include any of the same data discussed above with respect to authentication algorithm 170. When patient support apparatus 20 receives wall unit certificate 174, it analyzes the certificate 174 to determine if wall unit 60 is an authentic wall unit 60. This authentication analysis may include any of the same steps that server 84 performs when analyzing certificate 174, as discussed above with respect to algorithm 170. In general, certification 174 includes, if it is authentic, some kind of a shared secret or key phrase that is also known to patient support apparatus 20. The shared secret or key phrase may be built into patient support apparatus 20 and wall unit 60 during the times of their manufacture, and/or added thereafter through a configuration process. When patient support apparatus 20 receive the key phrase and/or shared secret, it checks memory 136 to see if the key phrase and/or shared secret matches with the authentic shared secret or key phrase. If there is a match with the key phrase and/or shared secret (and/or other information within certificate 174), control system 126 concludes that wall unit 60 is an authentic wall unit 60. If there is no such match, control system 126 concludes that wall unit 60 is not authentic.

If control system 126 concludes that wall unit 60 is authentic, it forwards its own patient support apparatus certificate 180 to wall unit 60 at step 194. Wall unit 60 carries out a similar authentication analysis with respect to patient support apparatus certificate 180 that patient support apparatus 20 carries out with respect to wall unit certificate 174. If wall unit 60 concludes from this analysis that patient support apparatus 20 is not an authentic patient support apparatus 20, wall unit 60 does not establish a communication session with patient support apparatus 20. If wall unit 60 concludes from this analysis that patient support apparatus 20 is an authentic patient support apparatus 20, wall unit 60 proceeds to send a session key to patient support apparatus 20 at step 196. The session key may be a Diffie-Hellman key message that includes a predefined portion and a random portion of data for use for that particular communication session. In response to receiving this message, patient support apparatus 20 sends a Diffie-Hellman key message back to wall unit 60 at step 198. This message may also include a predefined portion and a random portion for use during that particular communication session.

After the exchange of Diffie-Hellman messages at steps 196 and 198, wall unit 60 and patient support apparatus 20 establish a communication session with each other. This communication session involves one or more messages sent by wall unit 60 to patient support apparatus 20 at step 200, and/or it includes one or more messages sent by patient support apparatus 20 to wall unit 60 at step 202. Steps 200 and 202 may be repeated for as long as the communication session continues. The communication session may be terminated in the same manner discussed above with respect to algorithm 170.

Although algorithms 170 and 190 have been described above with respect to an authentication process between patient support apparatus 20 and a wall unit 60, it will be understood that either or both algorithms 170 and/or 190 may also be used by patient support apparatus 20 when attempting to establish a communication session with a tag 160 and/or a device 162 attached to a tag 160b.

It will also be understood that, in at least one embodiment, the authentication analyses discussed above are not performed by patient support apparatus 20 when it uses UWB transceivers 124 to determine the relative position of wall unit 60 and/or a tag 160. That is, patient support apparatus 20 is configured to determine this relative position information with a wall unit 60 and/or tag 160 regardless of whether or not the wall unit 60 and/or tag 160 are authentic. Indeed, in some embodiments, patient support apparatus 20 determines this relative position as a precursor for carrying out the authentication analysis. That is, as was discussed above, in some embodiments, control system 126 only proceeds to carry out an authentication analysis if the wall unit 60 or tag 160 are positioned within a threshold distance (range 156 or 158) of patient support apparatus 20, and does not carry out this authentication analysis if the wall unit 60 or tag 160 are not positioned within that threshold distance.

In addition, the encryption using the session key exchanged as result of the authentication analysis (algorithms 170 or 190) is only used for certain information exchanged between patient support apparatus 20 and wall units 60 or tags 160, and not used for other information exchanged between these devices. That is, control system 126, wall units 60, and tags 160 are configured to encrypt a first set of data that is communicated between themselves, and to not encrypt a second set of data that is communicated between themselves. In general, the first set of data includes data that is not used for determining the relative position of the patient support apparatus 20 with respect to the wall unit 60 and/or tag 160, and the second set of data includes the data that is used for determining the relative position of the patient support apparatus 20 with respect to wall unit 60 and/or tag 160. The first set of data may include a wide variety of non-location data, such as, but not limited to, sensor information, audio signals, commands, status data, and/or other data.

In at least one embodiment, control system 126 is configured such that it must first authenticate a wall unit 60a before it sends any audio signals from microphone 140 to the wall unit 60a, and/or before control system 126 sends any commands for any of the room devices 72, 74, or 78 to the wall unit 60a. Similarly, control system 126 may be configured such that it must first authenticate a wall unit 60a before it plays any audio signals received from the wall unit 60a on a speaker onboard patient support apparatus 20. The same is true for patient support apparatus status data (e.g. an exit alert, the position of one or more siderails 36, the state of onboard brake, the height of litter frame 28, etc.) that patient support apparatus 20 may communicate to wall unit 60a—i.e. such data is not communicate until after wall unit 60a has been authenticated. As was explained above, the post-authentication communications are encrypted with the session key that was established during the authentication process. In some embodiments, patient support apparatus 20 is configured to not encrypt communications with wall units 60b, even after control system 126 has authenticated the wall unit 60b.

In some embodiments, wall units 60 are configured to forward to patient support apparatus 20 data defining the threshold range 156. Still further, in some embodiments, control system 126 is adapted to use association thresholds 158 of different sizes (and/or shapes) depending upon the particular type of tag 160 and/or device 162 that a tag 160b is attached to. In such embodiments, tag 160 and/or device 162 sends data to patient support apparatus 20 indicating what type of tag 160 or device 162 it is, and control system 126 is configured to use this type data to determine the boundaries of association threshold 158. In these embodiments, control system 126 may further be configured to carry out the authentication analysis discussed above (algorithms 170 and/or 190) only if the wall unit 60 or tag 160 is positioned within the threshold range 156 or association threshold 158. It can therefore be seen that control system 126 is adapted, in at least some embodiments, to receive data from wall units 60 and/or tags 160 that defines, or is used to define, a condition that must first be met before control system 126 carries out the authentication analysis. The condition that must be met is the relative position of the wall unit 60 or tag 160 being within the corresponding threshold range 156 or threshold 158. After the condition is met, the authentication process takes place and, if successful, the subsequent communication session is encrypted with the session key shared during the authentication process.

After patient support apparatus 20 has authenticated a wall unit 60 or tag 160, it is configured, in at least some embodiments, to automatically determine what level of authorization the wall unit 60 or tag 160 has been assigned. This level of authorization may be based on an ID received from the wall unit 60 (e.g. wall unit ID 116) or an ID received from a tag 160. In such cases, memory 136 includes logic and/or data enabling control system 126 to determine the corresponding authorization level based on the received ID. Alternatively, control system 126 may be configured to forward the ID it receives to server 84 for server 84 to determine the corresponding authorization level. In either case, depending upon the authorization level, control system 126 is configured to carry out communications in different manners.

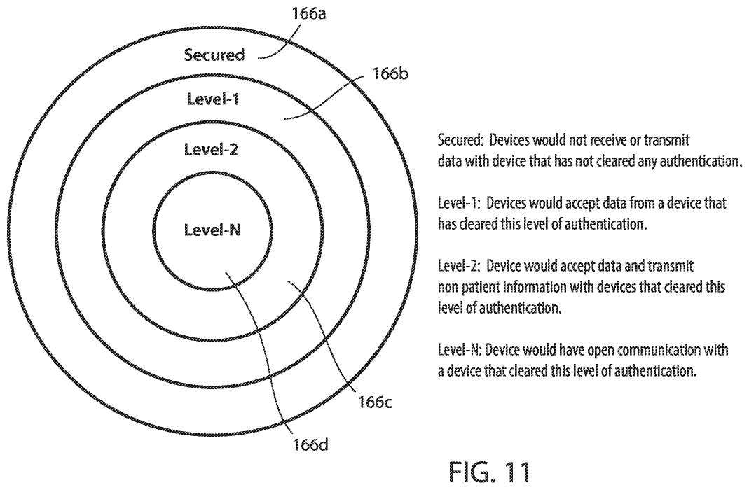

FIG. 11 illustrates one manner in which control system 126 may be configured to carry out communications with devices (wall units 60, tags 160, or devices 162) in different manners, depending upon the authorization level of the wall unit 60, tag 160, or device 162. As shown therein, control system 126 is configured to assign one of four different authorization levels 166a-d to the wall unit 60, tag 160, or device 162. A first authorization level 166a corresponds to communications that take place with a wall unit 60, tag 160, or device 162 that has not yet been authenticated, or that has failed the authentication analysis. With this level of authorization, patient support apparatus 20 does not share any information with the device or receive any information from the device (other than location information via transceivers 124, as was mentioned above). A second authorization level 166b corresponds to devices (wall units 60, tags 160, or devices 162) that have been authenticated, but that are considered to have a low level of authorization. With this level of authorization, control system 126 of patient support apparatus 20 is configured to accept data from the device, but to no send any data to the device.

A third level of authorization 166c (FIG. 11) provides a higher level of authorization than levels 166a or 166b. With the third level of authorization 166c, control system 126 of patient support apparatus 20 is configured to both accept data from the device, and to send non-patient data to the device. The non-patient data refers to data that doesn't relate directly to the patient, and/or that doesn't include any information that could be used to identify the patient. With the third level of authorization 166c, control system 126 is configured to not share any patient information with the device 60, 160, 162. A fourth level of authorization 166d provides the highest level of authorization for a device 60, 160, 162. With this fourth level of authorization 166d, control system 126 of patient support apparatus 20 is configured to exchange any data with the device 60, 160, 162 including patient data.

It will be understood that the authorization levels of FIG. 11 may be modified to include greater numbers of authorization levels or fewer numbers of authorization levels. Additionally, or alternatively, the rules of communication corresponding to one or more of these authorization levels may be varied from what has been described above and what is shown in FIG. 11. It will also be understood that the authorization levels of FIG. 11 apply, in some embodiments, to any communications carried out using Bluetooth transceiver 122 and only those communications of UWB transceivers 124 that are not used for determining the relative position of the wall unit 60 or tag 160. In other embodiments, the rules for the authorization levels 166 may apply to the transceivers 122, 124 in other manners. Still further, in some embodiments, any of the authorization rules may be applied to communications between Bluetooth transceiver 122 and a Bluetooth transceiver that is built into a medical device 162 to which a tag 160b is coupled.

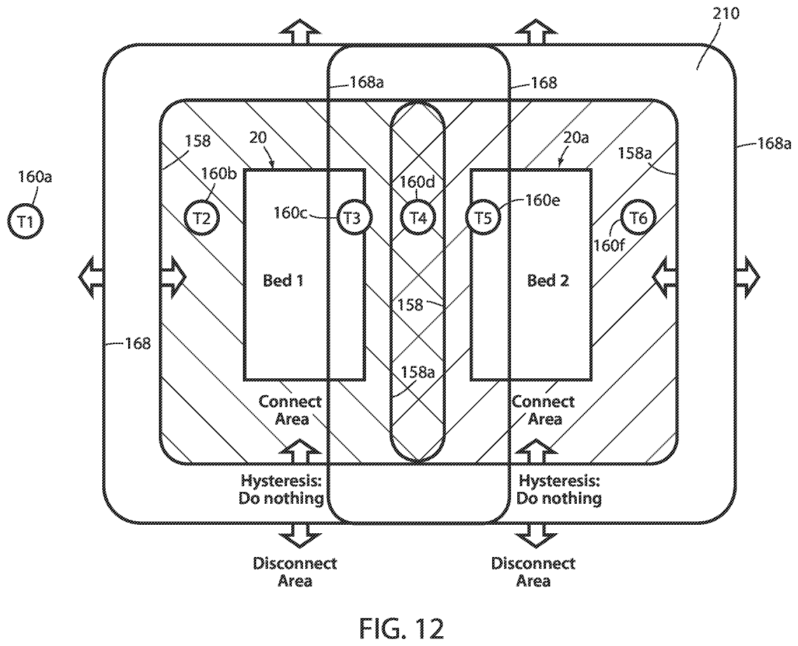

FIG. 12 illustrates one manner in which controller 134 and/or patient support apparatus server 84 may be configured to automatically determine what tags 160 to associate patient support apparatus 20 with, and what tags 160 to automatically disassociate patient support apparatus 20 from. As was described previously, each patient support apparatus 20 includes one or more association thresholds 158 defined about patient support apparatus 20. In the example shown in FIG. 12, there are two patient support apparatuses 20 and 20a, and each one has its own association threshold 158 and 158a, respectively. When controller 134 of patient support apparatus 20 detects a tag positioned inside of its association threshold 158, such as tag 160b, it either automatically associates the tag 160b with patient support apparatus 20, or it send information about the relative location of tag 160b to patient support apparatus server 84 and patient support apparatus server 84 associates tag 160b with patient support apparatus 20.

In some instances, such as shown in FIG. 12, the association threshold 158 of a first patient support apparatus 20 may overlap with the association threshold 158a of an adjacent second patient support apparatus 20a. In such situations, it is undesirable for both patient support apparatuses 20 and 20a to associate themselves with a tag 160, such as tag 160d, that is positioned within both association thresholds 158 and 158a. Such dual-association is improper because whatever device 162 that tag 160d is attached to is only being used with a single patient, and therefore should only be associated with a single patient support apparatus 20 or 20a.

In some embodiments, in order to help determine which patient support apparatus a tag 160 that is positioned within multiple association thresholds 158 should be associated with, such as tag 160d in FIG. 12, the controllers 134 of each patient support apparatus 20 and 20a are configured to automatically share with each other a list of the tags 160 that are currently positioned within their respective association thresholds 158 and 158a. The list may identify each tag 160 by a unique identifier that is transmitted by the tag 160 to the UWB transceivers 124 of each patient support apparatus 20. The list may be shared using direct patient support apparatus-to-patient support apparatus communication, such as via UWB transceiver(s) 124 and/or Bluetooth transceiver 122 (the latter of which is possible if the device 162 and/or tag 160 has a corresponding Bluetooth transceiver). Alternatively, this list may be shared by first sending it to patient support apparatus server 84, using network transceiver 94, which then forwards the list from one patient support apparatus to the other one. However shared, the list includes the identifier of each tag 160 that is positioned within association threshold 158 as well as the distance of each tag 160 from the patient support apparatus.

Once each patient support apparatus receives the list of tags 160 that are positioned within the other patient support apparatus's association threshold, each controller 134 of each patient support apparatus checks the received list against its own list and identifies any tags 160 that are common to both lists. In other words, controller 134 uses the lists to identify tags 160 that are on both lists. In the example shown in FIG. 12, tag 160d would be on both lists, and both controller 134 of patient support apparatus 20 and controller 134 of patient support apparatus 20a would identify tag 160d as being within the association thresholds 158 and 158a of both patient support apparatuses 20 and 20a.

In some embodiments, in order to resolve which patient support apparatus a tag like tag 160d (FIG. 12) should be associated with, the controllers 134 of each patient support apparatus determine which patient support apparatus the tag 160d is currently closer to and automatically associate the tag 160d with whichever patient support apparatus 20 or 20a it is currently closer to. Thus, in the example of FIG. 12, controller 134 of patient support apparatus 20 determines the distance between patient support apparatus 20 and tag 160d using its own UWB transceivers 134. It also looks at the distance between tag 160d and patient support apparatus 20a that was identified in the list it received from patient support apparatus 20a. It then compares the two distances and identifies which patient support apparatus (20 or 20a) is closer to tag 160d.

When determining the closer patient support apparatus (20 or 20a), each controller 134 may use a threshold distance that the tag 160d's position has to exceed in order to make the automatic association. The threshold, in some cases, is equal to, or greater than, the estimated level of accuracy of the position determinations carried out using UWB transceivers 124. For example, if controller 134 is able to determine the accuracy of the position of tag 160b down to six inches and controller 134 of patient support apparatus 20 determines that tag 160d is positioned four inches closer to patient support apparatus 20 than patient support apparatus 20a, it will not automatically associate tag 160d with patient support apparatus 20 because the four inches is less than the accuracy threshold of six inches. On the other hand, if controller 134 of patient support apparatus 20 determines that the position of tag 160d is closer to patient support apparatus 20 than to patient support apparatus 20a by, say, ten inches, controller 134 will automatically associate tag 160d with patient support apparatus 20.

In those situations where the controllers 134 of patient support apparatuses 20 and 20a detect a tag 160 that is equidistant to both patient support apparatuses 20 and 20a, or that is closer to one than the other but by less than the threshold mentioned above, either or both controller 134 may be configured to automatically display a message on their respective displays 52 informing the caregiver that the tag (e.g. tag 160d of FIG. 12) cannot automatically be associated with one of the patient support apparatuses 20 or 20a. The message may also instruct the caregiver to take one or more steps to manually associate the tag 160d with one of the patient support apparatuses 20 or 20a. In some embodiments, the manual step may involve having the caregiver physically move the tag 160d closer to whichever patient support apparatus 20 or 20a he or she wants the tag 160d to be associated with. Since the position of the tag 160d is repetitively being determined using UWB transceivers 124, the controllers 134 will see the position of tag 160d change such that it will approach one of the patient support apparatuses 20 or 20a to a greater extent than to the other one. Once the degree to which the tag 160d is closer to first patient support apparatus 20 than to second patient support apparatus 20a, or vice versa, exceeds the threshold mentioned above, controller 134 of the closer patient support apparatus 20 or 20a will automatically associate the tag 160d with itself. If the degree of closeness doesn't exceed the threshold, controller 134 will re-display, or continue to display, the message that it is unable to automatically associate the tag 160d with either patient support apparatus 20 or 20a.

Another method by which a caregiver can manually associate a tag 160 with a particular patient support apparatus 20 or 20a is to use near field transceivers that are built into patient support apparatus 20 and tag 160 (or the device 162 to which the tag 160 is coupled). When the tag 160 (or device 162) and patient support apparatus's near field transceiver are positioned within close proximity (e.g. several inches) of each other, the two exchange information that establishes that that particular tag 160 (or device 162) should be associated with that particular patient support apparatus (e.g. 20 or 20a in FIG. 12). Further details regarding the use of near field transceivers for associating objects with patient support apparatus 20 are disclosed in commonly assigned U.S. patent application Ser. No. 63/352,061 filed Jun. 14, 2022, by inventors Jerald Trepanier et al. and entitled COMMUNICATION SYSTEM FOR PATIENT SUPPORT APPARATUSES, the complete disclosure of which has already been incorporated herein by reference.

Another type of manual association process involves using scannable technology. For example, each tag 160 and/or device 162 may include a QR code, a bar code, or some other type of optical code that can be read by a corresponding scanner built into each patient support apparatus 20. In such situations, the caregiver simply scans the optical code of the tag 160 or device 162 with the scanner that is built into whichever patient support apparatus he or she wishes to associate the tag 160 or device 162 with. Thus, for example, if tag 160d of FIG. 12 should be associated with patient support apparatus 20, he or she scans tag 160d using the scanner of patient support apparatus 20. If tag 160d of FIG. 12 should be associated with patient support apparatus 20a, he or she scans tag 160d using the scanner of patient support apparatus 20a. In some embodiments, a scanner may be used that is not built into the patient support apparatuses. In such cases, each patient support apparatus also includes its own optical code, and the caregiver scans the optical code of both the tag 160d and the patient support apparatus with which it is to be associated.

Still another type of manual association process that may be implemented on patient support apparatuses 20 is for the caregiver to use one of the control panels 54 to manually instruct the corresponding patient support apparatus of a desired tag 160 association. In some embodiments, controller 134 is configured to display a list of tags 160 that are currently positioned within association threshold 158, along with a control that allows the user to confirm that they should be associated with that particular patient support apparatus 20. The control may be labeled “connect” or “associate” or the like, and when activated, it causes controller 134 to associate the corresponding tag 160 with that particular patient support apparatus 20. In some embodiments, controller 134 only displays the list of tags 160 on display 52 that are positioned within the association threshold 158 of multiple patient support apparatuses 20, thereby only allowing the caregiver to manually associate those tags whose association status may be questionable. In other embodiments, controller 134 may display the entire list of tags 160 on display 52 that are positioned within the association threshold 158 of that particular patient support apparatus 20, thereby allowing the caregiver to manually associate any of the tags 160 that are currently within association threshold 158.

In some embodiments, each patient support apparatus 20 automatically disassociates itself from a tag 160 if the tag 160 moves outside of association threshold 158. Alternatively, in some embodiments, controller 134 may use a separate disassociation threshold 168 for the automatic disassociation of a tag 160 from patient support apparatus 20. An example of such a disassociation threshold 168 is shown in FIG. 12. As shown therein, each patient support apparatus 20 and 20a includes a disassociation threshold 168 and 168a. If an associated tag 160 moves outside of disassociation threshold 168 or 168a of the patient support apparatus with which it is currently associated, the controller 134 of that patient support apparatus automatically disassociates the tag 160 from that patient support apparatus. Thus, for example, if tag 160a was previously associated with patient support apparatus 20, it would be automatically disassociated from patient support apparatus 20 in the position shown in FIG. 12 because, as shown therein, it is positioned outside of disassociation threshold 168. Similarly, if tag 160f had been previously associated with patient support apparatus 20, it would be automatically disassociated therefrom because it is outside of disassociation threshold 168. (Note, however, that if tag 160f had been associated with second patient support apparatus 20a, it would remain associated therewith because it is still positioned inside of disassociation threshold 168a of patient support apparatus 20a).

As shown in FIG. 12, the sizes of disassociations thresholds 168 and 168a are generally larger than the sizes of association thresholds 158 and 158a. This creates a hysteresis area 210 between the two thresholds 158 and 168 for each patient support apparatus. When a tag 160 is positioned in a hysteresis area 210 of a particular patient support apparatus, the controller 134 does not change the association or disassociation status of that tag 160. That is, if the tag 160 is currently associated with a particular patient support apparatus 20, it remains so when it moves into hysteresis area 210. Or if the tag 160 is currently disassociated with a particular patient support apparatus 20, it remains disassociated therefrom when it moves into area 210.

It will be understood that the diagram of FIG. 12 is a two-dimensional representation of multiple association and disassociation thresholds 158, 158a, 168, and 168a, and that, in an actual embodiment, thresholds 158, 158a, 168, and 168a may be three-dimensional. That is, the thresholds 158, 158a, 168, and 168a, in actual embodiments, will correspond to predefined volumes of space in which a tag 160 must be positioned in order for controller 134 to associate or disassociate a tag 160. It will also be understood that the size, shape, and/or position of thresholds 158 and/or 168 with respect to a patient support apparatus 20 may vary, and in some embodiments, thresholds 158 and/or 168 may be user-customizable, location-dependent, tag-dependent, and/or otherwise variable.

It will also be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A patient support apparatus comprising:
a support surface adapted to support a patient;
a plurality of locator nodes, each locator node including a location transceiver and a location controller, the location transceiver adapted to wirelessly communicate with a device positioned off-board the patient support apparatus and the location controller adapted to determine a distance between the location transceiver and the device;
an embedded network coupling together the plurality of locator nodes; and
a control system adapted to determine a location of the device relative to the patient support apparatus based on the distances determined by the location controllers, the control system further adapted to send time synchronization messages over the embedded network to the plurality of locator nodes, the time synchronization messages adapted to allow each of the location controllers to determine a synchronized time measurement.

2.-11. (canceled)

12. The patient support apparatus of claim 1 further comprising a transceiver adapted to wirelessly communicate with the device, and wherein the control system further adapted to determine if the location of the device meets a threshold condition, to receive identification data from the device, to perform an authentication analysis of the device if the threshold condition is met, and to not perform the authentication analysis of the device if the threshold condition is not met.

13. The patient support apparatus of claim 12 wherein the transceiver is a Bluetooth transceiver and each of the location transceivers are ultra-wideband transceivers.

14. The patient support apparatus of claim 13 wherein the control system is further adapted to determine an authorization level for the device based on the authentication analysis.

15.-20. (canceled)

21. The patient support apparatus of claim 14 wherein the control system is adapted to determine if the authorization level is a first or a second level, and if the authorization level is the first level, the control system is adapted to accept data from the device but not transmit any sensor or patient information to the device, and if the authorization level is the second level, the control system is adapted to both accept data from the device and to transmit sensor information, but not patient information, to the device.

22. The patient support apparatus of claim 21 wherein the control system is further adapted to determine if the authorization level is a third level, and if the authorization level is the third level, the control system is adapted to accept data from the device and to transmit patient information to the device.

23.-33. (canceled)

34. A patient support apparatus comprising:
a support surface adapted to support a patient;
a plurality of locator nodes, each locator node including a location transceiver and a location controller, the location transceiver adapted to wirelessly communicate with a device positioned off-board the patient support apparatus and the location controller adapted to determine a distance between the location transceiver and the device;
a transceiver adapted to wirelessly communicate with the device;
a control system adapted to determine a location of the device relative to the patient support apparatus based on the distances determined by the location controllers, the control system further adapted to determine if the location of the device meets a threshold condition, to receive identification data from the device, to perform an authentication analysis of the device if the threshold condition is met, and to not perform the authentication analysis of the device if the threshold condition is not met.

35. The patient support apparatus of claim 34 wherein the transceiver is a Bluetooth transceiver and each of the location transceivers is an ultra-wideband transceiver.

36. The patient support apparatus of claim 35 wherein the control system is further adapted to determine an authorization level for the device based on the authentication analysis.

37. The patient support apparatus of claim 35 wherein the identification data includes a device certificate, and wherein the control system is further adapted to transmit a patient support apparatus certificate to the device.

38. The patient support apparatus of claim 37 wherein the device certificate is received by the patient support apparatus in an encrypted form, and wherein the control system is adapted to encrypt the patient support apparatus certificate before sending the patient support apparatus certificate to the device.

39. The patient support apparatus of claim 38 wherein the control system is adapted to share a random session key with the device if the control system authenticates the device certificate, and to use the random session key for subsequent communications with the device.

40. The patient support apparatus of claim 39 wherein the control system uses the random session key for encoding communications with the device that are transmitted to the device via the transceiver.

41. The patient support apparatus of claim 40 wherein the control system is adapted to not use the random session key for encoding communications with the device that are transmitted to the device via the plurality of ultra-wideband transceivers.

42. The patient support apparatus of claim 34 further comprising a network transceiver adapted to communicate with a remote server, and wherein the identification data includes a device certificate, and the control system is further adapted to transmit the device certificate to the remote server as part of the authentication analysis.

43. The patient support apparatus of claim 36 wherein the control system is adapted to determine if the authorization level is a first or a second level, and if the authorization level is the first level, the control system is adapted to accept data from the device but not transmit any sensor or patient information to the device, and if the authorization level is the second level, the control system is adapted to both accept data from the device and to transmit sensor information, but not patient information, to the device.

44. The patient support apparatus of claim 43 wherein the control system is further adapted to determine if the authorization level is a third level, and if the authorization level is a third level, the control system is adapted to accept data from the device and to transmit patient information to the device.

45. The patient support apparatus of claim 34 wherein the device is a wall unit affixed to a wall of a healthcare facility.

46. The patient support apparatus of claim 45 further comprising a microphone adapted to convert voice signals from a patient onboard the patient support apparatus into audio signals, wherein the control system is further adapted to transmit the audio signals to the device only if the control system determines from the authentication analysis that the device is an authentic device.

47. The patient support apparatus of claim 34 further comprising an embedded network coupling together the plurality of locator nodes; and
wherein the control system is adapted to send time synchronization messages over the embedded network to the plurality of locator nodes, the time synchronization messages adapted to allow each of the location controllers to determine a synchronized time measurement.

48.-99. (canceled)
